(19) United States
(12) Patent Application Publication
(10) Pub. No.: US 2006/0062739 A1
(43) Pub. Date: Mar. 23, 2006

(54) Inhalable lidocaine formulation for treatment of asthma and for reducing the need for corticosteroids in asthmatic patients

(76) Inventors: Thomas Hofmann, Seattle, WA (US); Alan Bruce Montgomery, Medina, WA (US); Kevin Stapleton, Seattle, WA (US)

Correspondence Address: Hana Verny, Peters, Verny, Jones & Schmitt, LLP, 425 Sherman Avenue, Suite 230, Palo Alto, CA, 94306, US

(21) Appl. No.: 11/046,164

(22) Filed: Jan. 28, 2005

Related U.S. Application Data
(60) Provisional application No. 60/635,022, filed on Dec. 9, 2004.
(60) Provisional application No. 60/611,656, filed on Sep. 20, 2004.

(51) Int. Cl. A61L 9/04

(52) U.S. Cl. 424/45

(57) ABSTRACT

An inhalable lidocaine solution for treatment of asthma and a method for treatment of asthma with the reduced need for concurrent administration of oral corticosteroids in asthmatic patients. The lidocaine solution or lidocaine dry powder is delivered by an electronic nebulizer or by dry powder inhaler or dose meter one to several times a day.

This application is based on and claims priority of the Provisional applications Ser. No. 60/635,022, filed on Dec. 9, 2004, Ser. No. 60/611,656 filed on Sep. 20, 2004, and Ser. No. 60/539,836 filed on Jan. 27, 2004.

### BACKGROUND OF THE INVENTION

1. Field of the Invention

The current invention concerns an inhalable lidocaine formulation and a method for treatment of asthma with a reduced need for concurrent administration of oral, systemic or inhaled corticosteroids in asthmatic patients, as well as improved control of asthma symptoms and reduction of exacerbation. The method of the invention is also suitable for reduction of airway susceptibility to environmental stimuli, such as smoke, smog, dust, air pollutants and allergens. In particular, the invention concerns lidocaine formulated for use with an electronic nebulizer or dry powder or metered dose inhaler for targeted delivery of lidocaine into conducting and central airways as well as a method for treatment of asthma while at the same time reducing or eliminating the need for administration of oral or inhaled corticosteroids in asthmatic patients using said lidocaine formulation delivered by an electronic nebulizer or inhaler one to several times daily. The lidocaine formulation is administered in a daily dose from about 10 mg to 160 mg and delivered to the lungs. The lidocaine is formulated either as the lidocaine solution for inhalation or as a lidocaine dry powder. Lidocaine solution comprises 10 or 40 mg of lidocaine hydrochloride dissolved in one to three ml of normal or diluted saline per one dose delivery. The solution is optimized by adjustment of pH, osmolality, viscosity and anion concentration. The lidocaine solution is nebulized into an aerosol having a mass median aerodynamic diameter (MMAD) within a range from 3 μm to 10 μm and a substantially monodisperse particle spectrum using an electronic nebulizer preferably equipped with a vibrating perforated membrane. The dry powder is administered using a dry powder or meter dose inhaler. Lidocaine aerosolized according to the invention described herein is provided in the minimal effective dose and is deposited almost solely in the central airways without any substantial residue of lidocaine found in the oropharyngeal area, in bronchioli and alveoli of the lower lung, or systemically.

The method for treatment of asthma is more efficacious, faster and safer then the treatments described previously. Only the minimal amount of lidocaine is used and needed for abatement of asthmatic symptoms and the secondary undesirable symptoms such as bronchospasm, numbing of lips, tongue or pharyngeal region mucosa due to anesthetic properties of lidocaine or high systemic levels of lidocaine are eliminated or grossly reduced. The inhalable lidocaine solution is more efficacious in that a lesser amount of lidocaine is sufficient to achieve the same effect as the one previously achieved with larger dose. The lidocaine solution is nebulized using electronic nebulizer able to produce a substantially monodisperse particle spectrum resulting in a larger percentage of the lidocaine dose deposited in the central airways. Additionally, the need for concurrent treatment of asthma with corticosteroids is decreased or eliminated altogether following the administration of the lidocaine solution according to the invention.

2. Background and Related Disclosures

Asthma is a chronic disease of the conducting airways, in which inflammation is associated with bronchial reactivity. Inflammatory cells (primarily eosinophils and activated T-lymphocytes) infiltrate into the bronchial mucosa and submucosa, releasing mediators that increase mucosal permeability and production, cause smooth muscle contractility and increased reactivity of the airways to a wide variety of irritant stimuli (Am. Rev. Respir. Dis., 145:918-21 (1992) and J. Allergy Clin. Immunol., 88(6):935-42 (1992)).

Asthma is characterized by inflammation, bronchoconstriction, and frequent exacerbations that may require hospitalization. The mainstay of chronic asthma treatment is the administration of the β-agonists used to treat bronchoconstriction alone or in a combination with anti-inflammatory drugs, such as, for example, corticosteroids. Corticosteroids were introduced as an asthma therapy in 1950. They are administered parenterally, orally or by inhalation (J. Allergy, 21:282-287 (1950). Although their mechanism of action is not fully understood, corticosteroids have anti-inflammatory effects on asthmatic airways. Until today, they remain the most potent and consistently effective therapy for asthma (National Institutes of Health, 1-6 (2002), J. Allergy Clin. Immunol., 75:1-13 (1985).

Severe asthma, also called “steroid-dependent asthma” affects a relatively small proportion of asthmatics. Treatment options in this group are limited and consequently, chronic oral doses of corticosteroids are frequently used (Mt. Sinai J. Med., 70(3):185-90 (2003) and Thorax 48(2):139-41 (1993).

Chronic oral therapy with corticosteroids, however, is commonly associated with significant and severe side effects including truncal obesity, hypertension, glaucoma, glucose intolerance, acceleration of cataract formation, bone mineral loss, psychological effects, adrenal suppression and growth suppression in children. These side-effects have been linked to cortisol suppression, especially when dose levels of corticosteroids are high (Schimmer B P, Parker K L. Adrenocorticotropic Hormone: Adrenocortical Steroids and Their Synthetic Analogs; Inhibitors of the Synthesis and Actions of Adrenocortical Hormones. In Goodman and Gilmann's: The Pharmacological Basis of Therapeutics, 10^{th} Edition, New York, 1649-1678 (2001).

Attempts have been made to develop steroid-sparing drugs for asthma, such as, for example, cyclosporine (Allergy, 46(4):312-5 (1991)), methotrexate (J. Allergy Clin. Immunol., 88(2):208-12 (1991)), troleandomycin (J. Allergy Clin. Immunol., 78:36-43 (1986) and J. Assoc. Acad. Minor. Phys., 2(3):131-3 (1991)), oral gold and anti-immunoglobulin E (IgE) (Allergy Asthma Proc., 22(1):11-5 (2001) and J. Allergy Clin. Immunol., 81(1):6-16 (1998)).

Regrettably, all these new drugs have had only limited therapeutic success and to date, no satisfactory substitute for systemic oral corticosteroid therapy for asthma has been developed, identified or received regulatory approval.

It would therefore be advantageous to have available a method for treatment of asthma wherein the oral therapy with corticosteroids would be significantly decreased or altogether eliminated.

Lidocaine is a local and regional anesthetic currently approved for administration either as an injectable anesthetic for treatment of, for example, peripheral nerve, lumbar or caudal epidural block, as an intravenous infusion as an anti-arrhythmic agent or in topical preparations for dermal, ocular and mucosal numbing (O'Neil M J, Smith A, Heckelman P E, eds., The Merck Index: An Encyclopedia of Chemicals, Drugs and Biologicals, 13th ed., Whitehouse Station, N.J.: Merck & Co., Inc. (2001).

Topical lidocaine has been previously used prior to bronchoscopy to reduce airway reactivity and has been recommended as an aerosol for intractable cough and asthmatic tussive attacks (Chest, 105(5):1592-3 (1994), JAMA, 252(17):2456-7 (1984), Anaesthesia, 49(2):182 (1994) and Chest, 69(6):747-51 (1976).

Adverse events related to lidocaine are not uncommon and cases of anaphylactic reactions and acute respiratory distress syndrome (ARDS) upon pulmonary application have been reported Chest, 81(5):644-5 (1982), Chest, 83(3):585 (1983) and Chest, 83(6):933-4 (1983).

Adverse events associated with aerosolized lidocaine include numbing of lips, tongue and oral mucosa, and impaired gag reflex (Am. Rev. Respir. Dis., 122(6):823-8 (1980), Eur. J. Anaesthesiol., 17(11):672-9 (2000) and JAMA, 236(6):562 (1976).

The chronic topical application of aerosolized lidocaine to the airways used in previous studies was not associated with systemic toxicity (Mayo Clin. Proc., 71(4):361-8 (1996) and Ann. Allergy Asthma Immunol., 82(1):29-32 (1991).

The efficacy of chronic nebulized lidocaine was evaluated in studies in groups of adult and pediatric subjects with severe asthma at the Mayo Clinic in Rochester, Minn. These studies demonstrated a steroid-sparing effect and significant reduction in hospitalizations. No drug-related serious adverse experiences were reported and the majority of subjects with steroid dependent asthma were successfully weaned from oral corticosteroids (Int. J. Tuberc. and Lung Dis., 1:5, Suppl 1:S 32 (1997)).

In addition, lidocaine aerosol application to the airways attenuated the bronchoconstriction reflex provoked by inhalation challenges with histamine and methacholine, and challenges with hyperosmolar or hypoosmolar solutions, such as water. Am. J. Respir. Crit. Care Med., 154(4 Pt 1):885-8 (1996) and Chest, 72(4):429-38 (1977).

Upon aerosolization of 4% lidocaine (100 mg per dose) using the PARI LC PLUS™ jet nebulizer, oral numbing of lips, tongue and mucosa, along with an impaired gag reflex occurred for approximately 15 minutes following the inhalation of lidocaine (Am. J. Respir. Crit. Care Med., 163(5):A83 (2001). To overcome these reactions, it has been recommended that treated subjects not eat or drink for 1 hour after aerosol treatment. Under this regime, no adverse events related to impaired swallowing or reflux have been reported.

In recent years, use of lidocaine was proposed also for treatment and suppression of cough, particularly for instances where respiratory examination, i.e. bronchoscopy, were to ensue, which could be affected by a patient's cough (U.S. Pat. No. 6,362,197B1, JAOA, 98 (No 3):170-172 (1998), Chest, 105:1592-93 (1994), JAMA, 252 (No 17) 2456-2457 (1984), J. Canadian Assoc. Radiol., 22:199-200 (1971), Am. J. Emerg. Med., 19:206-207 (2001), Regional Anesthetics, 18:312-314 (1993), British J. Pharmacol., 138:407-416 (2003), J. Appl. Physiol., 74:1419-1424 (1993).

All previous publications of prior art have certain shortcomings in terms of safety and tolerability of inhaled lidocaine. Associated safety concerns with the administration of lidocaine are oropharyngeal numbing with loss of gag reflex, risk of aspiration of fluids and food, moderate to severe bronchospasm, and taste problems. In addition, the previously described treatments use a large amount of the lidocaine, are delivered slowly and inefficiently, need longer delivery times and are not efficient enough to provide rapid relief from asthma.

From the brief description above, it is clear that there is a continuous need for an effective, rapid and safe therapy for treatment of acute and chronic asthma. Such therapy would preferably comprise an inhalation of the aerosolized lidocaine delivering a therapeutically effective amount of 10 or 40 mg of lidocaine in one to three ml of saline by aerosolization in particle sizes being substantially within 3.5 and 10 microns directly to the endobronchial space of airways in a shortest possible time.

It is, therefore, a primary object of this invention to provide a method for treatment of asthma by providing a safe, physiologically acceptable and efficacious inhalable lidocaine for inhalation using a pure, preservative free lidocaine solution having pH between 5.5 and 7.5 which formulation contains a sufficient but not excessive concentration of lidocaine, can be efficiently aerosolized by nebulization using an especially adapted and modified electronic nebulizer into an aerosol having an MMAD within a range from 3.0 μm to 10 μm and a substantially monodisperse particle distribution, or a dry powder formulation with similar aerosol properties administered with a dry powder or metered dose inhaler, both well tolerated by patients.

All patents, patent applications and publications cited herein are hereby incorporated by reference.

### SUMMARY

One aspect of the current invention is an inhalable nebulized lidocaine for targeted delivery of lidocaine into conducting and central airways for treatment of asthma.

Another aspect of the current invention is an inhalable lidocaine solution or powder for treatment of asthma, said solution or powder nebulized using an electronic nebulizer or inhaler with a substantially monodisperse particle spectrum (GSD <1.7) into an aerosol with MMAD in the range of about 3.0 μm to about 10 μm.

Yet another aspect of the current invention is a method for treatment of asthma, said method comprising once, twice or several times a day administration of a nebulized lidocaine solution comprising 10 or 40 mg of lidocaine dissolved in about 1 ml of saline having pH between 5.0 and 7.5 in a total daily dosage from about 20 mg to about 80 mg predominantly into conducting and central airways, said solution nebulized into an aerosol with MMAD substantially in a range of from about 3.0 μm to about 10 μm, said aerosol delivered within 1 to 2.5 minutes.

Still yet another aspect of the current invention is a formulation of lidocaine dry powder whose particle size distribution has a MMAD between about 3.5 μm to about 10 μm and a substantially monodisperse, particle spectrum for efficient deposition of lidocaine into conducting and central airways.

Still another aspect of the current invention is a formulation comprising either about 10 or about 40 mg of lidocaine in a normal or diluted saline solution or other aqueous solvent containing chloride, wherein said formulation has a pH between 5.5 and 7.0, unbuffered, osmolality between 150 and 550 mOsm/kg, ion concentration between 31 and 300 mM of chloride as a permeant anion, viscosity smaller than 1.5 cp, which formulation is delivered by nebulization in about 1 ml of solution wherein the resulting aerosol has a MMAD between 3.5 μm and 10 μm and a relatively monodisperse particle spectrum and wherein said formulation is nebulized using an electronic nebulizer equipped with a vibrating perforated membrane.

Still yet another aspect of the current invention is a formulation comprising either about 10 or about 40 mg of lidocaine in a normal or diluted saline solution or other aqueous solvent containing chloride, wherein said formulation has a pH between 5.5 and 7.0, unbuffered, osmolality between 150 and 550 mOsm/kg, ion concentration between 31 and 300 mM of chloride as a permeant anion, viscosity smaller than 1.5 cp, which formulation is delivered by nebulization in about 1 ml of solution wherein the resulting aerosol has a MMAD between 3.5 μm and 10 μm and a relatively monodisperse particle spectrum and wherein said formulation is nebulized using an electronic nebulizer equipped with a vibrating perforated membrane, said formulation used for treatment of asthma and for reducing the need for oral corticosteroids in asthmatic patients.

Yet another aspect of the current invention is a method for treatment of asthma and for reducing or eliminating a need for simultaneous treatment of asthmatic patients with corticosteroids, said method comprising once, twice or several times a day administration of a nebulized lidocaine solution comprising 10 or 40 mg of lidocaine dissolved in about 1 ml of saline having pH between 5.0 and 7.5 in a total daily dosage from about 20 mg to about 80 mg predominantly into conducting and central airways, said solution nebulized into an aerosol with MMAD substantially in a range of from about 3.0 μm to about 10 μm, said aerosol delivered within 1 to 2.5 minutes.

Still yet another aspect of the current invention is a dry powder formulation comprising either about 10 or 40 mg of lidocaine, wherein said formulation is milled, spray dried or precipitated into a fine powder with a MMAD between about 3.5 μm and 10 μm and a relatively monodisperse particle distribution used for inhalation of the dry powder administered from one to four times per day not exceeding 160 mg per day.

Another aspect of this invention is a two-part reconstitution system comprising lidocaine in a dry or lyophilized powder form and a diluent stored separately until use.

### BRIEF DESCRIPTION OF THE FIGURE

FIG. 1 shows results of comparative studies of eight different nebulizers determining a total delivered dose of the drug and respirable dose of albuterol in time.

FIG. 2 is a graph showing a mean change baseline in morning peak expiratory flow following the administration of placebo, 1% lidocaine and 4% lidocaine inhalable solution.

FIG. 3 is a graph showing a mean change baseline in evening peak expiratory flow following the administration of placebo, 1% lidocaine and 4% lidocaine inhalable solution.

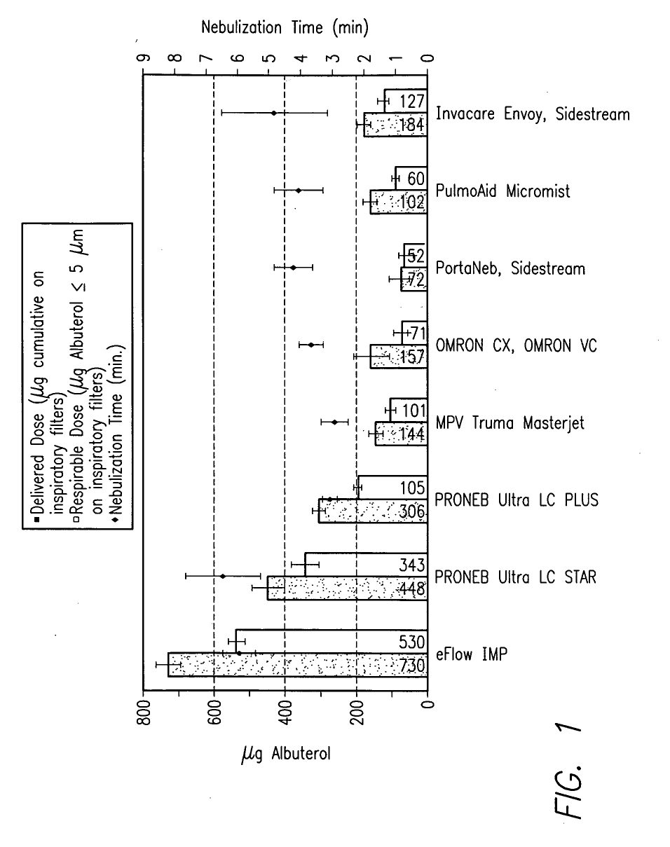

### DEFINITIONS

As used herein:

“MMAD” means mass median aerodynamic diameter.

“Normal saline” or “NS” means water solution containing 0.9% (w/v) NaCl.

“Diluted saline” means normal saline containing 0.9% (w/v) NaCl diluted into its lesser strength from about 0.04% to about 0.8%.

“Half normal saline” or “½ NS” means normal saline diluted to its half strength containing 0.45% (w/v) NaCl.

“Quarter normal saline” or “¼ NS” means normal saline diluted to its quarter strength containing 0.225% (w/v) NaCl.

“One tenth normal saline” or “ 1/10 NS” means normal saline diluted to its one tenth strength containing 0.09% (w/v) NaCl.

“One twentieth normal saline” or “ 1/20 NS” means normal saline diluted to its one tenth strength containing 0.045% (w/v) NaCl.

“Physiologically acceptable solution” means a saline diluted to between 1/10 NS and 1 NS or another aqueous solution comprising from about 31 to about 154 mM of chloride.

“Composition” means a lidocaine containing formulation which may additionally contain other components, such as excipients, diluents, isotonic solutions, buffers, etc.

“Formulation” means a specific composition formulated for specific use, such as for nebulization of lidocaine containing solution or nebulization of lidocaine dry powder.

“Lidocaine composition” or “lidocaine formulation” means a composition or formulation comprising an indicated amount of lidocaine.

“Central airways” means a section in respiratory tract defined by trachea, carina and bronchi.

“Carina” or “carina tracheae” means the ridge separating the opening the right and left main bronchi at their junction with the trachea.

“LSI” means lidocaine solution for inhalation.

“TOR” means total output rate.

“GSD” means geometric standard deviation.

“AE” means adverse event.

“SAE” means serious adverse event.

“AST” means aspartate aminotransferase.

“ALT” means alanine aminotransferase.

“ARDS” means acute respiratory distress syndrome.

“COPD” means chronic obstructive pulmonary disease.

“CS” means corticosteriod.

“ICS” means inhaled corticosteroid.

“OCS” means oral corticosteroids.

“FEF” means forced expiratory flow.

“FEV1” means forced expiratory volume in one second.

“FVC” means forced vital capacity.

“PEF” means peak expiratory flow.

“LFT” liver function test.

“LSI” lidocaine solution for inhalation.

“MDI” means metered dose inhaler.

### DETAILED DESCRIPTION OF THE INVENTION

The current invention concerns a discovery that specifically formulated and delivered inhalable lidocaine nebulized predominantly into particle sizes between about 3.0 μm and about 10 μm is safe and efficacious for treatment of asthma and, when used for treatment of asthma according to the invention, it does decrease or eliminate the need for a concurrent oral, inhalation or systemic treatment with corticosteroids in the asthmatic patients.

The treatment of asthmatic patients with lidocaine solution for inhalation (LSI) provides a means for weaning the patients from orally or systemically administered corticosteroids and thus provides opportunity to reduce the dose of orally administered corticosteroids while at the same time provides abatement of the asthmatic symptoms. A major benefit of the treatment of asthma with the LSI according to the invention is a reduction of the lidocaine dose into a nominal efficacious dose delivered in the smallest possible volume in a very short period of time, a reduction or elimination of oral or systemic corticosteroids, elimination of severe adverse effects caused by high doses of corticosteroids and an improvement of clinical parameters related to asthma in the asthmatic patients.

Consequently, the invention concerns an inhalable lidocaine composition for treatment of asthma comprising a minimal but efficacious amount of lidocaine and a method for treatment of asthma while decreasing amount of concurrently administered corticosteroids. The lidocaine composition is delivered to a patient's conducting and central airways by inhalation of a lidocaine dry powder or lidocaine solution for inhalation nebulized into an aerosol with a MMAD from about 3.0 μm to about 10 μm, preferably from about 4 μm to about 5 μm.

The current invention provides an efficacious, safe, nonirritating, physiologically acceptable and compatible inhalable lidocaine composition suitable for use in a method for treatment of asthma. The method provides for fast delivery, within 1-2.5 minutes, of efficacious amount of inhalable lidocaine. For aerosolization, lidocaine is delivered as a dry powder having particle size between 3.5 μm and 10 μm or as a solution comprising 10 or 40 mg of lidocaine dissolved in one ml of of saline, having pH between 5.5 and 7.5 and osmolality between 200 and 400 mOsm/kg. The solution is nebulized into an aerosol having a mass median aerodynamic diameter (MMAD) between 3.0 μm to 10 μm, using an electronic nebulizer able to aerosolize the lidocaine solution into particles of required sizes.

I. Asthma and the Current Treatment Thereof

Asthma is a chronic pulmonary disease characterized by reversible airway obstruction, airway inflammation and increased airway responsiveness to a variety of endogenous or exogenous stimuli.

Asthma is characterized by the airway obstruction due to a combination of spasm of the conducting airways mucosa, increase of mucus secretion, inflammation, infiltration of the airway walls with eosinophils and activated T-lymphocytes and injury and desquamation of the airway epithelium.

Treatment of asthma is a complicated management process including a drug therapy as well as an environmental control and elimination of asthma provoking symptoms. The drug therapy is divided into those for immediate symptoms relief, such as for example, β-agonists, that is β-adrenergic drugs such as albuterol, terbutaline, pributerol, metaproterenol and other known β-agonists, muscle relaxants, such as theophilline, or anticholinergic drugs, such as atropine and ipratropium which block cholinergic pathways that cause airway obstruction. Although these drugs have certain sometime severe side effects, due to a lack of better means for treatment, control and management of asthma, these drugs are still commonly used for treatment of asthma.

For treatment, management, control and prevention of chronic asthmatic symptoms, such as the lung inflammation, the preferential drug treatment is an oral or parenteral administration of corticosteroids. Corticosteroids have anti-inflammatory effects on asthmatic airways and are and remain the most potent and effective therapy for asthma. In cases of severe asthma, also called steroid-dependent asthma, patients treatment options are limited and these patients are typically treated with chronically administered oral doses of corticosteroids.

Chronic oral therapy with corticosteroids is known to be accompanied and associated with significant side effects including such severe symptoms as hypertension, glaucoma, glucose intolerance, acceleration of cataract formation, bone mineral loss, psychological effects, truncal obesity, supression of adrenal system and growth suppression in children. These side-effects have been linked with cortisol suppression, especially when dose levels of corticosteroids are high.

Attempts to achieve treatment of asthma without corticosteroids were widely unsuccessful although there are some indications that the oral administration of corticosteroids may be decreased when LSI in amounts from 160-640 mg daily is administered to patients with chronic asthma (Mayo Clin. Proc., 71:361-368 (1996)).

The new method for treatment, management and control of asthma provides a means to achieve such treatment with a substantially decreased or altogether eliminated administration of corticosteroids using minimal amounts of aerosolized lidocaine.

Using the current method, the asthma patients with increased sensitivity to environmental airway challenges from smoke, smog, dust, allergens and air pollution and patients with chronic asthma experience a substantial improvement of their symptoms when treated with 10 mg or 40 mg inhaled lidocaine at least one or twice daily with continuous tapering of oral corticosteroids to levels where the dose of the corticosteroids is lowered by at least half, or corticosteroids are altogether eliminated with no observable detrimental effects during such tapering or thereafter.

The clinical protocol for therapeutic use of inhalable lidocaine is based on following guidelines:

1) to deliver an effective and safe dose of lidocaine directly to the site of action in the lungs of asthmatic subjects;

2) to avoid significant systemic exposure by providing inhalable doses well below blood levels tolerated in other uses of lidocaine; and

3) to lower levels of oral corticosteroids without patient impairment.

The method comprises delivery of inhalable 1% or 4% lidocaine dissolved in saline, preferably in one ml of a normal or diluted saline (1.0 mL of 40 mg/mL solution) as an aerosol using the electronic nebulizer, such as for example, PARI eFlow electronic nebulizer, at least once, twice or several times daily. Orally delivered corticosteroids are slowly and continuously tapered to zero, until their level reaches 50% of the originally required dose or lower until the undesirable consequences occur.

The studies performed during development of this invention shows that LSI is well tolerated, safe and efficacious for treatment of asthma.

II. Conducting and Central Airways and Asthma

Conducting and central airways are comprised of trachea, carina and bronchi where bronchospasm and inflammatory processes observed during the asthma occur. The inhalation therapy of asthma thus targets the areas where the asthmatic reaction occur. Consequently, the lidocaine for inhalation is formulated in such a way as to be preferentially deposited in these three areas.

III. Lidocaine

Lidocaine is a local anesthetic known under the chemical name acetamide 2-(diethylamino)-N-(2,6-dimethylphenyl).

Lidocaine suitable for use in this invention is commercially available, for example from DSM Wyckoff, South Haven, Mich., and packaged by Cardinal Health Technologies-STW, Woodstock, Ill., as 1% (10 mg) or 4% (40 mg) lidocaine hydrochloride solution for intravenous use. Lidocaine solution for inhalation (LSI) is provided as a 1.0 mL sterile, preservative free, nonpyrogenic single dose ampule. The ampules contain either 10 or 40 mg of lidocaine hydrochloride, USP (1 mL 1% or 4% of lidocaine hydrochloride solution) having a pH range from 5.0 to 7.5. The sodium chloride content is 6.844 g/L of sodium chloride, USP, for 1% lidocaine and 0.351 g/L of sodium chloride, USP, for 4% lidocaine. The osmolality for both solutions is approximately 275-300 mOsm/kg.

IV. Lidocaine Inhalable Compositions

The current invention primarily concerns an inhalable lidocaine composition suitable for efficacious delivery of lidocaine into the central airways by nebulization of the lidocaine solution or a lidocaine dry powder.

In this invention, lidocaine solution for inhalation (LSI) is intended to be used in combination with an electronic nebulizer, preferably the one specifically modified and equipped with a vibrating perforated membrane, such as and preferably the PARI eFlow electronic nebulizer. Only in combination between LSI and the appropriate electronic nebulizer or inhaler will the advantages of this invention be valid and apparent. LSI is specifically formulated for inhalation, is preservative free and optimized regarding osmolarity, pH, anion concentration and viscosity, to be optimized for nebulization via the electronic nebulizer.

Lidocaine for inhalation (1% and 4%) may be delivered with or without pre-treating the patients with an inhaled β-agonist, such as for example, albuterol further protecting the lungs from bronchospasm.

The lidocaine solution for inhalation is preferably administered using the electronic nebulizer PARI eFLOW™, producing substantially monodisperse particles of between 3 and 10 μm. For nebulization of the lidocaine dry powder, the dry powder or metered dose inhalers that produce aerosols with a MMAD between about 3.5 μm and 10 μm are used. Such particle sizes are necessary for efficacious delivery of lidocaine into the central airways while minimizing the deposition of lidocaine anesthetic oropharyngeally and in lower lung.

Selection of the nebulizer for practicing this invention is very important and an indivisible part of the invention because most nebulizers are designed for use with medications for the treatment of lung diseases which need to be deposited in the peripheral airways.

A. Aerosolized Lidocaine Solution for Inhalation

Lidocaine composition for nebulization is formulated for most efficacious but safe delivery of aerosolized lidocaine to the lung central airways.

The lidocaine composition comprises 10 or 40 mg of lidocaine hydrochloride delivered in a total volume of 1 ml of saline for one inhalation dose. When formulated and delivered according to the method of the invention, it delivers a therapeutically efficacious dose of lidocaine to a target site of asthma in an amount of lidocaine sufficient to treat an asthmatic attack.

A combination of lidocaine composition with the improved means of delivery of said lidocaine composition using the electronic nebulizer that produces an aerosol with a substantially monodisperse particle spectrum, particularly the PARI eFlow nebulizer equipped with a vibrating perforated membrane, obtained from PARI GmbH, Munich, Germany, permits a delivery of substantially whole dose of lidocaine into conducting and central airways without any substantial deposition of lidocaine into oropharyngeal space, where it is known to cause local numbing and loss of gag reflex, or into the lower lungs where it could cause undesirable side effects and easily enter the systemic circulation.

Each dose of lidocaine solution contains a minimal yet efficacious amount of lidocaine of either 10 or 40 mg, per one dose, formulated in the smallest possible volume (1 ml) of saline having osmolality between 275 and 300 mOsm/Kg and pH between 5.0 and 7.5, preferably the pH of about 5.5-6. Lidocaine solution for inhalation is adjusted to permit generation of a lidocaine aerosol well tolerated by patients that minimizes the development of secondary undesirable side effects such as bronchospasm, loss of gag reflex, numbing and has a minimal oropharyngeal deposition.

1. Safety

Primary requirement for the aerosolized local anesthetic formulation is its safety. Safety is measured by the anesthetic effect of the local anesthetic asserted on the other areas of the respiratory tract, by its deposition in other areas of the respiratory tract than those where asthma occurs and by its numbing effect. Bronchospasm of the lung in asthma is a one of the most observable symptoms, as described amply in the literature, and is therefore important that the administration of lidocaine to the upper lungs does not aggravate bronchospasm due and related to any preservative contained in the inhalable lidocaine solution, or is not related to or caused by the particle sizes of the lidocaine aerosols. With a non-targeted delivery of lidocaine into the lungs, side effects such as systemic effects on the central nervous system, headache, tremor and dizziness are known to occur and are a measure of safety.

Since the lidocaine for inhalation is formulated to contain only a nominal amount of lidocaine and is delivered in particle sizes predominately deposited in targeted lung areas, the method for treatment of asthma is both safe and efficacious.

2. Efficacy

Efficacy is measured by the amount of the drug needed for asthma abatement, by the frequency of administration needed to suppress asthmatic attack, by the time necessary for delivery of the drug amount and by the percentage of the drug deposited in the specific target areas, namely in trachea, carina and bronchi as well as a lack of deposition in the other areas, namely in the upper airways, such as mouth, nose, larynx and pharynx and in the lower lungs, such as bronchioli and alveoli. Very importantly, efficacy is measured by the patient's tolerance to environmental challenges to the airways, and his/her tolerance to smoke, smog, dust, allergens and air pollution.

Main advantages of the current lidocaine formulation are its safety, its efficacy in asthma abatement without, or with decreased doses or concurrently administered oral corticosteroids, its lesser anesthetic effect, its lesser oropharyngeal deposition, its lack of bronchospasm, its faster delivery and its targeted dosing, practicality and convenience of use as well as its long shelf-life, storage and ease of administration and manipulation of the nebulization device. Because of its convenience, safety and practicality of the formulation and the nebulizer, the treatment may be provided in hospital setting, in the doctors office or at home.

Both the safety and efficacy requirements for aerosolized lidocaine have now been found to be met by the lidocaine formulation described herein.

3. Tolerability

The key parameters for airway tolerability of the lidocaine formulation for inhalation during aerosolization exposure which needs to be met are osmolality, pH, lidocaine concentration, ion concentration, viscosity and the absence of preservatives. These parameters are listed in Table 1, below.

TABLE 1
Tolerability Parameters of Inhalable Lidocaine

| | |
|---|---|
| Osmolality | >150-<550 mOsm/kg |
| Ion Concentration | >31-<300 mM permeant anion |
| pH | 5.5 to 7.0, unbuffered |
| Viscosity | <1.5 cp |
| Drug Concentrations | 1-4% lidocaine HC1 |
| Surfactants | None |
| Preservatives | None |
| Nebulization Time | <1-2.5 minutes |

As seen in Table 1, the lidocaine solution of the invention has osmolality between 150 and 550 mOsm/kg, ion concentration between 31 and 300 mM of the permeant anion, pH between 5.5 and 7.0 and viscosity lower than 1.5 centipoise. The lidocaine concentration is either 10 or 40 mg per one ml of saline. Other than saline, there are no other preservatives which could cause secondary side effects. Nebulization time for administration of one ml of the lidocaine solution is about 1-2.5 minutes when delivered with an electronic nebulizer on the output rate of the eFlow electronic nebulizer which has total output rate (TOR) higher or equal to 0.4 g/minute. When the output rate is about 0.5 g/minute, the delivery of 1 ml of the lidocaine formulation is shortened to less than 2 minutes.

From the above description is it clear that the lidocaine formulation for inhalation, as described herein, combined with the electronic nebulizer having the above described characteristics delivers the efficacious amount of lidocaine into lungs of the asthmatic patient within less then two minutes and at most at 2.5 minutes. The exposure of the patient to lidocaine is thus substantially shortened compared to all prior inhalation attempts with lidocaine and such treatment is, therefore, better tolerated.

4. Dosage of Lidocaine

The effective treatment of asthma requires a treatment regimen which provides sufficient amount of drug to suppress the asthma attack. Such regimen requires administration of an inhalable lidocaine one to up to four times or more a day. Most preferred dosing regimen for patient convenience is once, or preferably twice a day, however, because of a specific anesthetic effect of lidocaine asserts on the lungs and because of its relatively short life-time of about 2.5 hours, sometimes more than twice a day dosing is required for complete management of asthma. While typically for the treatment of asthma according to the invention a twice-a day regimen is sufficient, the lidocaine formulation may be safely administered four times a day or even more frequently, either in 10 or 40 mg dose.

A total daily dose of lidocaine is therefore set to be between either about 10 or about 160 mg per day administered in one or up to four doses of 10 or 40 mg per one dose. The total maximum recommended daily amount should typically not exceed about 200 mg.

Typically, the formulation and the electronic nebulizer are selected to provide at least about 25-40%, preferably higher than 50%, efficacy of lidocaine delivery to the conducting and central airways. Thus, with 10 or 40 mg dose, between 2.5 to 4 mg is delivered if the dose is 10 mg/ml. When the dose is 40 mg, the actually delivered amount of lidocaine into lungs is between 10 and 16 mg of during each administration. Three mg of lidocaine delivered to the lung has been found to be efficacious in patients suffering from seasonal non-severe asthma. For suppression of severe chronic asthma, the 16 mg dose delivered as a 40 mg dose/1 ml of saline according to the invention, is very efficacious, and since it can be delivered in a very short period of time of less then 2 minutes, it is effective and was found to be void of any severe undesirable effects, such as numbing of the oropharyngeal area or increased systemic plasma levels. In no instance should one dose exceed 80 mg lung dose.

Determination of the effective dosage of administered lidocaine and the regimen used for treatment of each patient depends on the responsiveness of the individual patient to the treatment. The ultimate decisive factor is the expected level of lidocaine after aerosolization in the area where asthmatic reactions occur. In addition, the lung dose is also correlated with lidocaine plasma levels. The optimal range of lidocaine in 1 ml of plasma immediately after nebulization should be in the 20-500 ng/mL range. Thus, the frequency of the administration is correlated with the effectiveness of administered lidocaine.

The new mode of administration permitting a noninvasive administration of small yet effective amounts of lidocaine directly into conducting and central airways provides substantial improvement compared to all previously known methods used for delivery of nebulized lidocaine.

5. Effect of pH on Lidocaine Aerosol Formulation

The pH of the nebulized formulation containing lidocaine is an important feature for treatment of asthma. Consequently, the saline solution used for preparation of lidocaine aerosol has certain requirements. Such aerosol has to provide osmolality between 275 and 300 mOsm/kg and not to affect the optimal pH range, which is from 5.5 to 7.0, preferably between pH 5.5 and 6.5.

The control of pH of the LSI formulation is necessary for efficacious delivery of the nebulized lidocaine. When the lidocaine nebulized aerosol is either more acidic or basic, that is outside of the range of pH given above, it can cause bronchospasm in central airways and exacerbate the asthma, which would be counterproductive. Although the safe range of pH is relative and some patients may tolerate a mildly more acidic aerosol, others will experience bronchospasm. Any aerosol with a pH of less than 4.5 typically induces bronchospasm. Aerosols with a pH between 4.5 and 5.5 will cause bronchospasm occasionally. Testing of a lidocaine aerosol discovered that an aerosolizable lidocaine formulation having a pH between 5.5 and 7.0 is well tolerated and safe. Any aerosol having pH greater than 8.5 is to be avoided as the lung epithelium is unable to buffer larger amounts of alkaline aerosols. Aerosol with pH below 4.5 and over 8.5 results in lung irritation accompanied by severe bronchospasm, exacerbated asthma, and inflammatory reactions.

The target pH of the formulation should be between 5.5 and 7.0. Even though some studies have shown that acidic solutions as low as pH 2.0 can be tolerated in the airways, many cases of acid-evoked bronchospasm have been reported. Also, extremes of pH even in the presence of chloride anion proved important stimuli when experimentally inducing asthma. Therefore, it is recommended that for treatment of asthma the pH of a nebulized solution be above pH 5.0. Likewise, it is known that a pH above 9.0 is toxic to mammalian tissue. Therefore, a solution that mimics physiological pH parameters, that is pH around pH 5.5-7.0 is least likely to cause irritation.

For these reasons as well as for the avoidance of bronchospasm, asthma or inflammation in patients, the optimum pH for the lidocaine aerosol formulation was determined to be between pH 5.5 to pH 7.0 with tolerable pH between pH 5.0 and 7.5. Consequently the lidocaine aerosol formulation is adjusted to pH between 5.5 and 7.0 with preferred pH range from about 5.5 to 6.5. Most preferred pH range is from pH 5.5 to pH 6.

6. Effect of Salinity on the Lidocaine Formulation

Patients suffering from acute or chronic asthma, particularly those with chronic asthma, have increased sensitivity to various chemical agents and have high incidence of bronchospastic asthmatic incidents. Since this method is designed for treatment of asthma, the salinity of the local anesthetic solutions is very important.

The airways of the patient suffering from asthma are particularly sensitive to hypotonic or hypertonic and acidic or alkaline conditions and to the excess but also the absence of a permanent ion chloride. Any imbalance in these conditions or a presence of chloride above certain values leads to bronchospastic or inflammatory events and/or exacerbated asthma which greatly impair treatment with inhalable formulations. All these conditions prevent efficient delivery of aerosolized lidocaine into the central airways. The bronchospasm and inflammatory clinical manifestations of the irritated airways are extremely undesirable for a method of treatment of asthma according to this invention.

For treatment of asthma with lidocaine solution for inhalation, the use of an aqueous solvent without providing certain degree of osmolality to meet and emulate physiological conditions found in healthy lungs is impossible.

Consequently, a certain amount of the chloride anion is needed for successful and efficacious delivery of aerosolized lidocaine and such amount is much more specific than amounts provided and typically used for aerosols of other compounds.

Bronchospasm accompanying asthma does not respond to the same osmolality of the diluent for aerosolization, however, such bronchospasm can be sufficiently controlled and/or suppressed when the osmolality of the diluent is in a certain range. Preferred solution for nebulization of lidocaine which is safe and has airways tolerance has a total osmolality between 275 and 300 mOsm/kg with a range of chloride concentration of between 31 mM and 300 mM. The given osmolality controls bronchospasm and the chloride concentration, as a permeant anion, contributes to the control of asthma.

Normal saline (NS, 0.9%) contains 154 mM of chloride whereas 31 mM of chloride corresponds to about 0.2% normal saline. Lidocaine salt is manufactured as lidocaine HCl. Higher concentrations of lidocaine solution for inhalation therefore needs lesser addition of NaCl, in order to reach the 150 mM chloride content.

It has now been discovered that lidocaine may be efficaciously delivered into central airways when dissolved in normal saline, that is saline containing 0.9% of sodium chloride, or diluted, that is lesser than normal saline. The 1/20 N saline permits and assures a delivery of lidocaine into central airways and in some cases permits better particles deposition and treatment of asthma.

Consequently, the formulation for lidocaine aerosol of the invention comprises either about 10 or about 40 mg, preferably about 40 mg, of lidocaine dissolved in 1 ml of a normal or a diluted saline to from about 1/20 normal saline (NS) to about and at most to 1 normal saline solution.

The lidocaine formulation containing about 10 mg of lidocaine per 1 ml of 0.2 NS has an osmolality of about 290 mOsm/l. Such osmolality is within a safe range of aerosols suitable for administration to patients suffering from asthma and also those patients with chronic asthma.

Since the delivery of lidocaine formulated as described herein is much more efficacious, much lower total dose of lidocaine is needed to achieve complete and fast suppression of asthma. Typically, about 40 mg total dose of lidocaine dissolved in 1 ml of solution is sufficient in suppressing the asthma when delivered with an electronic nebulizer, preferably PARI eFlow electronic nebulizer modified as described above.

7. Osmolality

The osmolality of an aerosolized solution is directly related to the initiation of bronchoconstriction during inhalation. A study of 9 mild asthmatics has shown that hyperosmolar solutions, such as 4% sodium chloride (1232 mOsm) or hypo-osmolar solutions, for example distilled water, (0 mOsm) induced bronchoconstriction when nebulized aerosol was inhaled. Conversely, iso-osmolar solutions (308 mOsm) did not induce bronchoconstriction. Therefore, an isotonic solution, such as 0.9% sodium chloride, would be the least likely solution to cause bronchoconstriction.

Likewise, asthma is regularly induced by inhalation of solutions with osmolality <100 or >1100 mOsm/kg. Nebulized antibiotic solutions have an osmolality >150-<550 mOsm/kg and contain permeant ions, such as chloride, in concentrations >31 mM-<300 mM in order to be readily tolerable in the airway.

Another point of consideration is the effect of nebulization on the osmolality of the solution. During nebulization, the osmolality can increase 11% to 62%, as compared with the pre-nebulization value. The peak increase in osmolality is typically observed between 10 and 15 minutes of nebulization. This rise in osmolality may be explained by the mechanisms of nebulization. In a jet nebulizer, the aerosol is produced by the fluid shearing in a high velocity stream of dry gas. After primary droplet generation, water evaporates from the surface of the aerosol droplets to humidify the air thereby increasing the osmolality in the droplet. Approximately 99% of the droplets then return to the reservoir causing a continuous increase in the concentration of the solute in the liquid remaining in the nebulizer and a continuous increase in the osmolality of the aerosol droplets. Because of this increase in osmolality restricting the nebulization time to no more than 10 minutes was recommended and, of course, the shorter the time of nebulization, the lesser increase in osmolality occurs.

Consequently, since when using the PARI e-Flow electronic nebulizer or one of the other similarly equipped electronic nebulizers, the time of nebulization is shortened to 1-2 minutes, this concentration effect is negated and no drug concentration occurs during nebulization.

8. Ion Concentration and Permeability

The absence of permeant anion in ultrasonically nebulized solutions is a stimulus for asthma even under iso-osmolar conditions, and the amount of asthma is directly proportional to the concentration of permeant anion. Therefore, not only is the ion concentration important for airway tolerability, but the type of ion present must also be considered.

Inhalation of a solution with osmolality between 225 and 616 mOsm/kg induces asthma when the chloride concentration is less than 31 mM. Chloride was found to be an ideal permeant ion, with its presence mitigating some of the adverse effects caused by the hypertonicity of nebulized solutions. A chloride concentration between 31-300 mM was found to be optimal. If the ion used is not chloride, the selected alternative should freely permeate the respiratory mucosa.

Examples of salts that produce suitable permeant anion and can be thus used as a substitute of the sodium chloride are calcium chloride, choline chloride, lysine monohydrochloride, potassium chloride, sodium chloride, sodium bromide and sodium iodide.

However, while it is possible to use these substitutes, for the purpose of this invention, the sodium chloride anion is most preferable.

9. Viscosity

The rate of nebulization and particle size distribution is directly proportional to the viscosity of the solution, as the rate of nebulization and particle size decrease as the viscosity increases. Concentrations of antibiotic solutions that produced viscosities greater than 1.5 cp were found to have a dramatic impact on the nebulization rate. Consequently, the viscosity of the lidocaine solution for inhalation should be kept near 1.5 centipoise (cp).

10. Additives

As already stated above, the lidocaine solution for inhalation is preservative free and preferably no other additives are used.

Any use or intent to use additives will require careful consideration concerning its effects on the airway tolerability and toxicity of the solution.

11. Preferred Aerosolizable Lidocaine Formulations

The lidocaine aerosolizable formulations comprise lidocaine in amount about 10 or about 40 mg in about 1 ml of saline, having pH adjusted to between 5.0 and 7.5, said formulation delivered by aerosolization using an electronic nebulizer equipped with a vibrating perforated membrane.

The preferred formulation of the current invention is a formulation comprising either about 10 or about 40 mg of lidocaine dissolved in about 1 ml of saline, having pH adjusted to between 5.5 and 7.0, delivered by nebulization in an aerosol having a mass median aerodynamic diameter (MMAD) between 3.0 μm and 10 μm, wherein said formulation is nebulized using an electronic nebulizer.

The most preferred formulation of the current invention comprises about 40 mg dose of lidocaine dissolved in about 1 ml of saline, having pH adjusted to between 5.5 and 7.0, delivered by nebulization in aerosol particles having the mass median aerodynamic diameter predominantly between 3.0 and 10 Am, preferably 4 μm and 5 μm, wherein said formulation is nebulized using a PARI eFlow electronic nebulizer preferably the one equipped with a vibrating perforated membrane.

Table 2 shows parameters for two specific lidocaine formulations.

TABLE 2
Parameters 1% and 4% Lidocaine Solution For Inhalation

| | 1% Lidocaine | 4% Lidocaine |
|---|---|---|
| | HCl | HCl |
| [Cl−] mM | 153 | 153 |
| Osmolality mOsm/kg | 291 | 286 |
| pH | 6.4 | 6.2 |
| ppm (Na+) | | 160 |
| surface tension | | 58.32 |
| dynes/cm = mN/m | | |
| viscosity cps | 1.39 | 1.33 |
| Density | 1.00315 | 1.00239 |
| % label claim | 99 | 100 |

All formulations are designed to be well tolerated and able to be reliably and completely nebulized to aerosol particles within the respirable size range of 3.0 μm to 10 μm, preferably within 4 Mm and 5 μm, deposited rapidly and predominantly in the central conducting airways.

The doses are designed to contain as much as, but not more than, the necessary amount of a most active form of lidocaine to prevent and treat severe asthma and tussive asthmatic attacks.

Patients can be sensitive to pH, osmolality, viscosity, and ionic content of a nebulized solution. Therefore these parameters are adjusted to be compatible with lidocaine chemistry and still tolerable to patients.

The formulation of the invention is nebulized into an aerosol with characteristics optimizing a delivery of the drug into the central airways where asthmatic reactions occur.

For efficacious delivery of lidocaine to the lung central airways in an aerosol particle, the formation of an aerosol having a mass median aerodynamic diameter (MMAD) between 3.0 μm to 10 μm, preferably between about 4 μm and about 5 μm, is necessary. The formulated and delivered amount of lidocaine for treatment of asthma must effectively target the lung conducting and central airways. The formulation must have a smallest possible aerosolizable volume able to deliver an effective dose of lidocaine in the shortest possible time. The formulation must additionally provide conditions which would not adversely affect the functionality of the central airways. Consequently, the formulation must contain enough of the drug formulated under the conditions which allow its efficacious delivery while avoiding numbing of upper respiratory tract and deposition in lower areas of lung not affected by asthma. The new formulation according to the invention meets all these requirements.

B. Lidocaine Dry Powder Composition

An alternative way to deliver inhalable lidocaine is by way of dry inhalable powder.

The lidocaine may be administered to the central airways in a dry powder formulation for efficacious delivery of the anesthetic into the central airways using dry powder or metered dose inhalers as an alternative to a liquid formulation delivered via nebulilizer.

A dry powder formulation has potency, on a mass basis, which allows such alternative delivery of lidocaine as a dry powder using dry powder inhaler. A sufficiently potent formulation of lidocaine provides a dry powder which can be advantageously delivered by dry powder inhaler or by metered dose inhaler. For delivery of dry inhalable powder, lidocaine is milled, precipitated, spray dried or otherwise processed to particles that when emitted from the dry powder inhaler forms an aerosol with a mass median aerodynamic diameter between about 3.5 μm and 10 μm.

Dry powder formulation comprises from either 10 or preferably 40 mg of lidocaine. For dry powder formulation of the invention, lidocaine is processed to a powder that when emitted from the dry powder inhaler has a mass median aerodynamic diameter ranging from 3.5-10 μm, preferably from about 4 μm to about 5 μm. Examples of powder processing technologies include, but are not limited to media milling, jet milling, spray drying, lyophilization or particle precipitation techniques.

In this aspect, the invention provides a sufficiently potent formulation of lidocaine formulated as a dry powder for use in a dry powder inhaler or metered dose inhaler such that when the powder is emitted from the device, the resulting aerosol has a MMAD between about 3.5 μm and 10 μm, preferably about 4 μm to about 5 μm. Such a delivery system is practical and convenient because it does not require any further handling such as diluting the dry powder or filling a nebulizer. Further, it utilizes small and fully portable inhalers.

The dry powder formulation is thus practical and convenient for ambulatory use because it does not require dilution or other handling, it has an extended shelf-life and storage stability and the dry powder inhalation delivery devices are portable and do not require large attachments needed by aerosol nebulizers.

All techniques suitable for preparation of dry inhalable powders and any and all improvements thereof as well as any dry powder inhaler are intended to be within the scope of the invention.

C. Shelf-Life and Storage

Stability of the formulation is another very important issue for efficacious formulation. If the drug is degraded before nebulization, a smaller and undetermined amount of the drug is delivered to the lung thus impairing the treatment efficacy. Moreover, degradation of stored lidocaine may generate materials that are poorly tolerated by patients.

The dry form of lidocaine has at least 2 years long shelf life. Lidocaine solution provided in examples has a shelf life given by the manufacturer.

A formulation for aerosolization can be provided as two separate components, one containing a dry or lyophilized lidocaine or a salt thereof and a second containing an appropriate diluent such as 0.1 to 0.9 N saline, as described above. The formulation is reconstituted immediately prior to administration. This arrangement prevents problems connected with the long-term stability of lidocaine in aqueous solvents.

According to the invention, lidocaine for aerosolization is preferably formulated in a lyophilized dosage form intended for use as a dry powder for reconstitution before inhalation therapy. The formulation of lidocaine can be aseptically prepared as a lyophilized powder either for dry powder delivery or for reconstitution and delivery, or as a frozen solution, a liposomal suspension, or as microscopic particles. The storage suitability of the formulation allows reliable reconstitution of the formulated lidocaine suitable for aerosolization.

The liquid form of lidocaine (1% and 4%) is conveniently supplied in 1 ml “Blow-Fill-Seal” vial, made of polyethylene plastic material. Specifically, the plastic to be used is low density polyethylene (LDPE) commercially available from Huntsman Rexene 6010. Overwrap is Flexicon Flexi-2114.

The material chosen for storage avoids absorption of lidocaine to the plastic walls of the vial (which is common with other plastics), and the 1 ml fill volume provides a safe and efficacious amount of drug, along with patient-convenient use of the plastic vial. In the described vials, with an aluminum overwrap, lidocaine 1% and 4% are stable for at least 9 months and 12 months, respectively, at room temperature, and no loss of strength occurs. At accelerated conditions (40° C. and 75% relative humidity), both formulations remained active for at least 6 months.

V. Administration of Local Anesthetics by Inhalation

Local anesthetics are administered by nebulization using the electronic nebulizer using the inhalable lidocaine formulation according to the invention as described above.

A. Two Modes of Inhalable Administration

Administration of inhalable lidocaine according to the invention is achieved either with lidocaine aerosol or with inhalable dry lidocaine powder, as described above.

In alternative, the drug may be prepared and stored as a powder and dissolved in saline just before administration, or directly administered as a powder.

B. Frequency of Dosing

The frequency of dosing is dependent on the severity of asthma and other conditions, such as other underlying diseases, for example, chronic obstructive pulmonary disease, lung cancer, emphysema, pneumonia, common cold or flu. A treatment regimen provides for one to four, preferably two, times a day administration of the inhalable lidocaine. A most preferred dosing regimen for patient's convenience is once or twice a day dosing, however, because of the rapid lidocaine asthma suppressing effect, and because of its relatively short life-time of about 8 hours (plasma half life of lidocaine upon aerosolization is 2.5 hours), more often dosing may be required in multiple administrations for complete asthma suppression.

In patients with severe asthma, the frequency of dosing may be increased to about four times a day, or even more frequently, if needed on as needed basis providing only such amount of lidocaine as necessary to suppress asthma is used.

The current daily dose can be as small as 10 mg. The typical upper limit is 200 mg of lidocaine per day delivered in two to four administrations with preferred upper limit of 160 mg. Typical and preferred range for one aerosol dosage is 10 or 40 mg in one ml administered twice a day or 10 mg administered three or four times per day. For dry powder inhalation, the dose for one administration is, typically, between about 5 and 20 mg per one dose and at maximum can reach 200 mg per one dose.

Aerosolization of lidocaine comprises nebulization of the lidocaine solution for inhalation using electronic nebulizers able to produce particle sizes in sizes predominantly between 3.0 and 10 μ and deliver 1 ml of lidocaine solution in less than 2.5 minutes, preferably in less than 2 minutes. Those electronic nebulizers that are portable are preferred for ambulatory treatment.

A dry powder inhalation, as the second mode of administration of the inhalable lidocaine, utilizes the lidocaine formulated as a lidocaine dry powder formulation. In this instance, lidocaine is delivered into the central airways space using dry powder or metered dose inhalers. The lidocaine potency, determined on a mass basis, allows the inhalation of lidocaine powder, as an alternative mode of administration to the aerosol. Dry powder inhalation is most efficacious, practical and economical when administered doses contain about 10 or 40 mg. The frequency of dosing is typically twice a day, but may happen three or four times a day. The invention also includes only one, or more than four times dosing regimen when necessary, as this regimen depends on the need and conditions of the patient.

The invention provides a sufficiently potent formulation of lidocaine in a form of dry powder delivered as metered dose inhalation of lidocaine particles processed into a powder such that when emitted from the inhaler forms an aerosol with a MMAD within a range of about 3.5 μm to 10 μm. Such dry powder delivery is possible and preferable particularly for ambulatory inhalation as it simplifies the delivery process. The drug delivery is convenient because it does not require any further handling such as diluting the dry powder or mixing the powder with a solvent, etc. Additionally, the dry powder formulation has longer shelf life than the liquid lidocaine formulation for aerosolization.

Severely asthmatic patients, for example, may be able to withstand only one inhalation at a time but could repeat this inhalation of small amount of lidocaine every two, three or four hours to obtain sufficient level of lidocaine to suppress asthma without causing or worsening bronchospasm.

VI. Devices for Delivery of Aerosolized Lidocaine

A primary requirement of this invention is to deliver lidocaine must efficiently to the central airways in a most rapid and economic way. Drug delivery to the lungs is a function of the size distribution of the inhaled aerosol, the delivery system, and the drug content of the particles. The effects of these aspects are well documented in the literature. A good review is contained in “The Mechanics of Inhaled Pharmaceutical Aerosols” by W. H. Finlay, Academic Press, (2001).

A. Nebulizers

The selected nebulizer must be able to efficiently aerosolize the formulation which has salinity, viscosity, osmotic strength, and pH adjusted according to the invention as to permit generation of lidocaine aerosol that is therapeutically effective and well tolerated by patients. The electronic nebulizer must be able to handle the formulation having a smallest possible aerosolizable volume and still be able to deliver effective dose of lidocaine to the site of the action. Additionally, the aerosolized formulation must not impair the functionality of the upper airways or lower lining spots must minimize undesirable side effects, and the delivered amount of lidocaine must be efficacious for treatment of severe and persistent asthma.

The inability of certain nebulizers to nebulize therapeutic quantities of drugs into uniform predetermined particle size aerosols is well known. For efficacious delivery of lidocaine a range of aerosolized particles with a MMAD needed to deliver the drug to the central airways only, the site of the asthma reactions, is between 3-10 μm. Many commercially available nebulizers are able to aerosolize large volumes of the solution with an aim to deliver at least 10% of the volume to the lung by producing around 90% of large aerosol particles above 10 μm with a very large number of particles being in the range of 50-100 μm. These nebulizers are inefficient and not suitable for delivery of lidocaine according to this invention.

The composition of the invention described above provides the drug formulated in a solution permitting delivery of a therapeutically efficient but nominal amount of the drug, provided that the aerosol generated by the nebulization meets criteria required for such efficient delivery. The electronic nebulizer which aerosolizes the formulation of lidocaine with substantially uniform particle sizes in times shorter than 2.5 minutes, according to the invention, is inseparable from the specific lidocaine solution.

There are quite a few nebulizer types currently commercially available. None of them but the electronic nebulizers is suitable for practicing this invention. Moreover, even among the electronic nebulizers, only those able to deliver 1 ml of lidocaine solution in time period shorter than 2.5 minutes, preferably below 2 minutes, are suitable. The most preferred nebulizers is PARI eFlow modified with a vibrating perforated membrane. Electronic nebulizer is defined as one of the nebulizers from the group described below.

Most pharmaceutical aerosols have particle size ranges between 1 μm and 100 μm as this size range has the best balance of inhalability and ability to transport drug. Within this range, smaller particles tend to deposit deeper in the lungs, and larger particles tend to deposit in the mouth and throat. Conversely, small particles contain much less drug (mass increases as the cube of the diameter) meaning that the time to deliver an efficacious dose to the lungs is much longer with smaller particles.

For deposition in the conducting and central airways, a particle size of approximately 4.5 microns is optimal. The advantage of the electronic nebulizers referred to herein e.g. PARI eFlow nebulizer, modified to be equipped with a vibrating perforated membrane over other nebulizers, is that the particle size distribution is adjusted and tuned to produce substantially all particles with the optimal size and deliver the drug as quickly as possible.

The most currently available pharmaceutical nebulizers produce polydisperse aerosols. Polydisperse aerosols consist of many particle sizes and consequently, the aerosols that are more polydisperse tend to deposit the particles over a wider region of the respiratory tract with a lesser dose of the drug deposited to the targeted area. The aerosol produced by the PARI eFlow is monodisperse, producing an aerosol with particle sizes having a geometric standard deviation (GSD) smaller than 1.7. Consequently, the majority of the aerosol particles are of the sizes between 3 and 10 with a large portion of these particles having a MMAD between 4 and 5 μm.

The advantage of using an electronic nebulizer that produces an aerosol with the preferred characteristics is that more drug is deposited to the site of action, that is to central airways with lesser residue deposited to the mouth and throat where it would cause numbing or in the lower lungs where it could enter the systemic circulation.

Although the dry powder and metered dose inhalers are suitable and contemplated to be used for delivery of the dry powder, an electronic nebulizer is preferable over these inhalers as there is no ballistic component of the aerosol exiting the device to cause excessive deposition in the mouth and throat. It also does not require the patient to achieve the high inhalation flow rates required to efficiently use many dry powder inhalers.

The main advantage of the electronic nebulizers such as PARI eFlow over other nebulizers is the speed of delivery. The PARI eFlow can produce aerosol much faster than other nebulizers, decreasing the treatment time substantially. Also, in comparison to other nebulizers, the PARI eFlow has much smaller drug residue left in the device after the treatment (residual volume), increasing the efficacy of the delivery and decreasing the cost of the therapy.

By combining all these aspects, the PARI eFlow or another comparable electronic nebulizer can deliver a dose of medication to the lungs 2-10 times faster than jet nebulizers, and 10-30 times faster than some other nebulizers while decreasing side effects due to aerosol not being deposited at the site of action.

The electronic nebulizer generally and PARI eFlow nebulizer particularly is, therefore, most preferred primarily on the basis of allowing the formation of lidocaine aerosol having a mass medium average diameter predominantly between 4 μm to 5 μm and a substantially monodisperse particle spectrum.

This aspect fo the invention is of great importance because for treatment of severe and persistent cough or for treatment of tussive attacks or episodes, the delivered amount of lidocaine must be efficacious and the delivery must be fast to avoid development of bronchospasm due to anesthetic properties of lidocaine. The selected nebulizer thus must be able to efficiently aerosolize the formulation which has salinity, osmotic strength, and pH adjusted according to the invention as to permit generation of lidocaine aerosol that is therapeutically effective and well tolerated by patients. The electronic nebulizer must be able to handle the formulation having a smallest possible aerosolizable volume and still be able to deliver effective dose of lidocaine to the site of the action. Additionally, the aerosolized formulation must not impair the functionality of the upper airways or lower lining spots and must minimize undesirable side effects.

The inability of certain nebulizers to nebulize therapeutic quantities of drugs into uniform predetermined particle size aerosols is well known. For efficacious delivery of lidocaine a range of aerosolized particles with MMAD needed to deliver the drug to the central airways only, the site of the cough receptors is between 3 μm-10 μm. Many commercially available nebulizers are able to aerosolize large volumes of the solution with an aim to deliver at least 10% of the volume to the lung by producing around 90% of large aerosol particles above 10 μ with a very large number of particles being in the range of 50-100 μm. These nebulizers are inefficient and not suitable for delivery of lidocaine according to this invention.

Previously, certain types of nebulizers, such as jet and ultrasonic nebulizers, have been shown to be able to produce and deliver aerosols with MMAD of between 1 μm and 5 μm. These aerosols might be optimal for treatment of pulmonary bacterial, viral or parasitic infections residing in the lower lungs, however, they are not sufficiently efficacious and selective in producing particles which deposit efficiently in the conducting and central airways. Additionally, these nebulizers typically need larger volumes to administer sufficient amount of drug to obtain a therapeutic effect. Typically, for example, the jet nebulizers are only about 10% efficient under clinical conditions. The amount deposited and absorbed in the lungs is thus a fraction of the 10% in spite of the large amounts of the drug placed in the nebulizer.

There are quite a few nebulizer types currently commercially available. None of them but the electronic nebulizers are suitable for practicing this invention. Moreover, even among the electronic nebulizers, only those with a specific parameters are suitable for practicing this invention. The electronic nebulizer best suitable for practicing the current invention must be able to deliver about at least 70% but preferably 90% or more of lidocaine or lidocaine-like compound dissolved in about 1-3 ml, preferably about 1 ml, of solvent in time period shorter than 2.5 minutes, preferably around 1-1.5 minutes. The most preferred electronic nebulizer is PARI eFlow™ nebulizer preferably the one modified with a vibrating perforated membrane.

B. Nebulizers—Comparative Efficacy

Several commercially available nebulizers were tested for their suitability in practicing this invention. Results of these studies are summarized in Tables 3 and 4.

Tables 3 and 4 show results of comparing efficacy of various types of nebulizers to deposit the drug in lungs in vivo.

TABLE 3

| | | Delivered | | |
|---|---|---|---|---|
| | Time/ | Dose | Respirable | |
| NEBULIZER | Min. | (μg) | Dose (μg) | Efficacy % |
| eFlow | 6 | 730 | 536 | 100% |
| Pro Neb Ultra LC Star | 6.5 | 445 | 343 | 61% |
| Pro Neb Ultra LC Plus | 3.5 | 306 | 195 | 42% |
| MPV Truma Masterjet | 3.3 | 144 | 101 | 20% |
| OMRONCX: OMRONVC | 3.9 | 157 | 71 | 21.5% |
| Porta Neb Sidestream | 4.5 | 71 | 53 | 9.7% |
| Pulmo Aid Micromist | 4.2 | 162 | 90 | 22% |
| Invacare Envoy Side | 5 | 184 | 127 | 25% |
| Stream | | | | |

Table 3 shows a time in minutes it takes various nebulizers to deposit a respirable dose of the drug.

As seen from results in Table 3, only the eFlow nebulizer was able to deliver 100% of the respirable drug dose in 6 minutes. All other nebulizers delivered between 9.7 and 61% of the drug.

As seen in Table 4, the 6 minutes time was set for delivery of the same respirable dosages as seen in Table 3.

TABLE 4

| | Time/ | | Delivered | | Respirable |
|---|---|---|---|---|---|
| NEBULIZER | Min. | | Dose | μg | Dose |
| eFlow | 6 | 730 | 100% | 536 | 100% |
| Pro Neb Ultra LC | 6 | 411 | 56% | 317 | 59% |
| Star | | | | | |
| Pro Neb Ultra LC | 6 | 524 | 72% | 334 | 62% |
| Plus | | | | | |
| MPV Truma Masterjet | 6 | 262 | 35% | 184 | 34% |
| OMRONCX: OMRONVC | 6 | 242 | 33% | 109 | 20% |
| Porta Neb | 6 | 192 | 26% | 71 | 13% |
| Sidestream | | | | | |
| Pulmo Aid Micromist | 6 | 231 | 31.5% | 128 | 24% |
| Invacare Envoy | 6 | 220 | 30% | 152.4 | 28% |
| Sidestream | | | | | |

When the time of nebulization was set to be 6 minutes, as seen in Table 4, again only the eFlow nebulizer delivered 100% of the respirable dose. All other nebulizers delivered much lower dose of the drug with percentage of the drug delivery only between 13-62% during the same time.

When the same nebulizers were evaluated in in vivo deposition studies, a lung deposition of approximately 40% of the total dose was seen when the electronic nebulizer was used. This was a marked increase in delivery efficiency compared to approximately 12% of the lung deposition of the dose administered with the jet PARI LC PLUS nebulizer.

Additionally, the oropharyngeal deposition is estimated at 5 to 10%, which is substantially lower than that of jet nebulizers (e.g., approximately 16% with the jet PARI LC PLUS nebulizer). Further, the PARI eFlow nebulizer's output of 8 to 10 μL/sec enables delivery of drug material 2-4 times faster than the PARI LC PLUS nebulizer. The basic performance specifications for the eFlow nebulizer are presented in Table 5.

TABLE 5
Performance Specifications for PARI eFlow Nebulizer

| NEBULIZER | Value | |
|---|---|---|
| Mass Median Diameter (MMD) | 3.3-3.8 | μm |
| Geometric Standard Deviation (GSD): | approx. 1.6 | μm |
| Total Output Rate (TOR) | >0.4 | g/min |

| | |
|---|---|
| Residual Volume | negligible* |

The PARI eFlow nebulizer is designed to aerosolize the whole 1 ml of the lidocaine solution placed in the device with allowance for a maximum of 150 μl of precipitation remaining on the device walls.

The combination of these benefits results in a treatment time of one tenth to less than half of current therapies, and potentially as low as 1-1.5 minutes per nebulization of 1 mL of LSI solution.

TABLE 6
Phaser Nebulizer Performance Tests In vitro

| | Value | |
|---|---|---|
| Phaser with 14.6% Lidocaine | | |
| Lidocaine Concentration | 146.5 | mg/ml |
| Actuation volume | 16 | μl |
| Filter deposition | 2.03 ± 0.08 | |
| Mg Particles 2.1-9 μm | 89% | |
| Phaser with 17.5% Lidocaine | | |
| Lidocaine Concentration | 175 | mg/ml |
| Actuation volume | 16 | μl |
| Filter deposition | 2.46 ± 0.18 | mg |

| | |
|---|---|
| Particles 2.1-9 μm | 72% |

In testing other nebulizers, seen in Table 6, two lidocaine solutions for inhalation, containing 14.6% and 17.5%, at 28.3 L/min, R=585 Ohm-m) were nebulized in vitro with the Battelle HH5 Phaser nebulizer. The particle sizes of the aerosol as well as deposition of the drug on a filter corresponding to inhaled dose were measured upon multiple actuations.

Results of the test with 17.5% lidocaine (175 mg/ml, 16 ul per actuation, nominal dose of 2.8 mg), show deposition of the drug on inhalation filters between 2.15 and 2.67 mg (mean 2.46±0.18 mg, RSD 7.16%) per actuation. The particle size distribution of all particles was between 2.1 and 9 μm with 72% of particle size distribution having sizes between 3.65 and 5.07 μm.

Results of the test with lidocaine 14.6% (146.5 mg/ml, 16 ul per actuation, nominal dose of 2.34 mg), show depositon of the drug on inhalation filters between 1.86 and 2.13 mg (mean 2.03±0.08 mg, RSD 3.85%) per actuation. The particle size distribution of all particles was between 2.1 and 9 μm with 89% of particles having sizes between showed 3.69 and 4.13. These studies show that the Battelle HH5 Phaser nebulizer could potentially be used for the current invention, however, there could be certain loss of the drug due to certain percentage of the particles being smaller than 3 μm.

Preferable electronic nebulizers are those electronic nebulizers that can produce aerosols with MMAD between about 4 and 5 with a relatively monodisperse particle spectrum (GSD <1.7). Examples of suitable electronic nebulizers are Aerogen Aeroneb Pro, Aerogen AeroNeb Go, Batelle White Phaser and its derivatives, Boehringer Spiromat, and preferably the PARI eFlow nebulizer. All these nebulizers can be used in practicing this invention.

The most preferred is the PARI eFlow nebulizer manufactured by PARI GmbH of Starnberg, Germany, equipped and modified with a vibrating membrane. However, it is to be understood that while preferred, the PARI eFlow is only one of the possible electronic nebulizers suitable for use in this invention and all other electronic nebulizers are contemplated to be within the scope of this invention.

Comparative study of the PARI eFlow electronic nebulizer delivering 1 ml, 2 ml and 3 ml doses of lidocaine or a lidocaine-like compound vis-a-vis a time of delivery, is shown in Table 7.

TABLE 7
Drug Delivery by the Modified PARI eFlow Nebulizer

| | Fill Volume | | |
|---|---|---|---|
| | 1 mL | 2 mL | 3 mL |
| Number of PARI eFlow tested | 1 | 9 | 1 |
| Number of tests | 2 | 18 | 2 |
| Nominal Drug Dose (mg) | 92 | 184 | 276 |
| DD (mg/drug/insp. filter) ± SD | 48.9 | 98.3 ± 2.6 | 148.8 |
| DD (%/drug/insp. filter) ± SD | 53.2% | 53.4 ± 1.4% | 53.9% |
| DDR (mg drug/min) | 22.1 | 22.5 | 24.4 |
| FF (droplets <5.8 μm) | 82.0% | 82.0% | 82.0% |
| measured by LD | | | |
| RD (mg drug) | 40.4 | 81.2 | 122.6 |
| RDDR (mg drug/min) | 18.3 | 18.6 | 20.2 |
| Nebulization time (min) | 2.21 | 4.37 ± 0.60 | 6.09 |

IMP = improved with a vibrating perforated membrane.

In Table 7, the PARI eFlow nebulizer was used to determine the efficacy of the drug delivery. The study was designed to compare nebulization of the nominal drug dose, 92 mg in 1, 184 mg in 2 ml and 276 mg in 3 mL of the solvent. Both the delivered dose (DD) and the respirable dose (RD) are expressed in mg of the drug. Additionally, the drug (mg) delivered per 1 minute (DDR and RDDR) and nebulization were determined. Results seen in the nebulization time column shows that respirable dose 40.4 mg can be delivered in 2.21 minutes, 81.2 mg can be delivered in 4.37 minutes and 122.6 mg of the drug can be delivered by 6 minutes long nebulization.

Results seen in Table 7 clearly show that the use of the PARI eFlow electronic nebulizer results in a significant improvement of the drug delivery rate and that the time of the drug delivery can be substantially shortened while the efficacy of the delivery is not affected by such time shortening.

B. Dry Powder Inhalers

Dry powder is administered as such using devices such as dry powder or meter dose inhalers which deliver the dry powder directly to the lungs.

For use in dry powder inhalers, the lidocaine or a lidocaine-like compound is formulated as a dry powder, as described above, in dosages from 1-100 mg, preferably from 10-50 mg. The particle sizes of the powder are such that when the powder is emitted from the inhaler, it forms an aerosol with a mass median diameter of between about 3.5-10 μm, preferably substantially between 4 μm and about 5 μm.

C. Efficacy of Lidocaine Nebulization

As described above, the selection and choice of the nebulizer greatly affects efficacy of the delivery of the inhalable lidocaine or a lidocaine-like compound.

A combination of an aerosol formulation of lidocaine or a lidocaine-like compound and a nebulizing device significantly enhances the efficiency and speed of lidocaine or a lidocaine-like compound administration.

Currently, for example the average time for administration of inhaled lidocaine or a lidocaine-like compound solutions using other formulations and nebulizers is 10-20 minutes per dose. Since, at this time, no safe and convenient plastic vial for packaging and storage of the lidocaine solution is available, patients need to use glass vials of i.v. lidocaine, assure that there are no preservatives or contaminants in the formulation, extract a defined amount of lidocaine from the vial by use of a syringe, and inhale via jet nebulizer. Such inhalation typically requires at least 10-20 minutes with a preparation time for inhalation adding another 10 minutes.

The time required for the currently available treatments results a significant loss of the drug, loss of the time, places unnecessary burden on the patient and contributes to reduced compliance with the daily regimen.

Furthermore, the nebulizer systems used previously for lidocaine administration are less efficient than new electronic devices. Using these nebulizers, the total deposited dose of drug in the lung is in the 12 to 15% range, at maximum. Approximately 30% of the dispensed drug remains in the nebulizer at the end of treatment and of the portion that is aerosolized about 30% is emitted as the particles which are too large or too small to reach the central airways. Oropharyngeal numbing, impairment of the gag reflex, cough, shortness of breath and breathlessness caused by a deposition of the drug in the peripheral and/or upper lungs and other systemic side effects are the consequences of these treatments.

The novel electronic nebulizer, with an output of 8 to 10 microliters/seconds, or 0.48 to 0.60 ml/minute, is capable of delivering drug material 2 to 30 times faster than the prior nebulizers. Furthermore, the novel nebulizer is able to aerosolize more than 90% of the dispensed dose. As a result, administration of a specifically designed formulation of lidocaine or a lidocaine-like compound using the electronic nebulizer leads to substantial improvement in delivery of the drug to the central airways, in a shorter time required for delivery and, depending on the final concentration of lidocaine or a lidocaine-like compound in the inhalable solution, reduces treatment time to as little as one to two minutes.

VII. Treatment of Asthma

Asthma is a chronic pulmonary disease characterized by reversible airway obstruction, airway inflammation and increased airway responsiveness to a variety of endogenous or exogenous stimuli. The airway obstruction in asthma is due to a combination of spasm of the conducting airways mucosa, increase of mucus secretion, inflammation, infiltration of the airway walls with eosinophils and activated T-lymphocytes and injury and desquamation of the airway epithelium.

Consequently, the treatment and/or management of asthma tends to be complicated.

A. Treatment of Asthma with Inhalable Lidocaine

Treatment of asthma is a complicated management process including a drug therapy as well as an environmental control and elimination of asthma provoking symptoms. The drug therapy includes β-agonists, muscle relaxants, anticholinergic drugs and, of course, corticosteroids.

For treatment, management, control and prevention of chronic asthmatic symptoms, such as the lung inflammation, the preferential drug treatment is an oral or parenteral administration of corticosteroids. Corticosteroids have anti-inflammatory effects on asthmatic airways and are and remain the most potent and effective therapy for asthma. In cases of severe asthma, also called steroid-dependent asthma, patients treatment options are rather limited and these patients are typically treated with chronically administered oral doses of corticosteroids.

Chronic oral therapy with corticosteroids is known to be accompanied and associated with significant side effects including such severe symptoms as hypertension, glaucoma, glucose intolerance, acceleration of cataract formation, bone mineral loss, psychological effects, truncal obesity, suppression of adrenal system and growth suppression in children. These side-effects have been linked with cortisol suppression, especially when dose levels of corticosteroids are high.

Attempts to achieve treatment of asthma without corticosteroids were so far unsuccessful although there are some indications that the oral administration of corticosteroids may be decreased when lidocaine in amounts from 160-640 mg daily is administered to patients with chronic asthma (Mayo Clin. Proc., 71:361-368 (1996)).

The new method for treatment, management and control of asthma provides a means to achieve such treatment with a substantially decreased or altogether eliminated administration of corticosteroids using minimal amounts of aerosolized lidocaine.

Using the current method, the asthma patients with increased sensitivity to environmental airway challenges from smoke, smog, dust, allergens and air pollution and patients with chronic asthma experience a substantial improvement of their symptoms. When treated with 10 mg or 40 mg inhaled lidocaine at least one or twice daily with continuous tapering of oral corticosteroids to levels where the dose of the corticosteroids is lowered by at least half, or when corticosteroids are altogether eliminated, the asthmatic symptoms decrease significantly with no observable detrimental effects during such tapering or thereafter.

The method of the invention provides safe, fast and efficacious treatment of asthma in the asthmatic patients and additionally it leads to decreased need or complete elimination of a concurrent oral or systemic treatment with corticosteroids.

The method for treatment of asthmatic patients with lidocaine solution for inhalation (LSI) provides a means for weaning the patients from orally or systemically administered corticosteroids and provides opportunity to reduce the dose of orally administered corticosteroids while at the same time provides abatement of the asthmatic symptoms.

A major benefit of the treatment of asthma using the current invention is a reduction or elimination of oral or systemic corticosteroids, elimination of severe adverse effects caused by high doses of corticosteroids and an improvement of clinical parameters related to asthma in the asthmatic patients.

A recently concluded Phase II clinical study described in Example 4 provides evidence that such treatment is successful with no significant adverse events related to study drug (lidocaine) or to the device (PARI eFlow nebulizer). The study also demonstrated the nebulizer's safety, efficiency of drug delivery, reliability and convenience resulting in high patient compliance with the study protocol. These results support the conclusion that the electronic nebulizer, particularly PARI eFlow is a reliable and efficient device for delivery of lidocaine in nominal dosages for efficacious treatment of asthma.

B. Two Modes of Inhalable Treatment for Asthma

A method for treatment of seasonal asthma and severe asthma comprises administration of lidocaine in inhalable form whether by aerosol or as a dry powder, two to four times a day. The lidocaine daily dose is 160 mg/day, 10 or 40 mg/ml for aerosol and from 10 to 200 mg daily dose of dry powder administered in a dose of 10-40 mg/one treatment. The lidocaine dosage and dosing frequency depends on the type of asthma, severity thereof, age of the patient, the conditions of the patient, etc.

The dry powder formulation suitable for treatment of asthma comprises administration of powder in an amorphous or crystalline state in particle sizes between 3.5 and 10 microns in mass median average diameter necessary for efficacious delivery of lidocaine into the conducting space. The dry powder formulation is delivered one to four (or more) times daily, preferably twice daily. The dry powder formulation is temperature stable and has a physiologically acceptable pH of 5.5-7.5, preferably 5.5 to 7.0, and an over five year long shelf life.

C. Treatment of Asthma and Other Diseases

Aerosol therapy of this invention is particularly useful for treatment of patients suffering from asthma accompanying other pulmonary diseases and is especially suitable for treatment of patients with severe asthma, chronic obstructive pulmonary disease (COPD), lung neoplasia, chronic bronchitis, gastroesophageal reflux, sarcoidosis, etc. There is also a number of patients suffering from chronic asthma resulting from a viral infection of the upper airways, i.e. post infectious asthma, who will benefit from the treatment of the current invention.

It has also been discovered that inhalable lidocaine provides successful treatment for asthma appearing in patients with cystic fibrosis, bronchiectasis or other suppurative pulmonary disease. Treatment of these conditions with aerosolized lidocaine has been successful in improving the disease by suppressing the asthmatic attacks.

D. Advantages of Inhalable Lidocaine

Lidocaine possesses several features that make it very attractive for administration to patients.

The first of these features stems from its mechanism of action, which involves modulation of nerve conductivity, and of airways smooth muscle control, by inhibition of uptake transporters and numbing.

Practical advantages are the conveniently provided 1 ml (10 mg or 40 mg; 1 or 4%) lidocaine vials, and the brevity of each treatment in about 2-2.5 minutes, including setup.

Medically, the advantages of the current invention are substantially improved safety, efficacy, tolerability, and targeted dosing with the eFlow.

VIII. In vivo Testing

The conditions requiring particular attention are seasonal asthma and severe asthma.

In order to determine if an appropriately formulated lidocaine for aerosolization could become effective for treatment of severe and persistent asthma, the treatment with aerosolized lidocaine was initiated and tested in asthmatic patients. The clinical treatment and results obtained with an aerosolized lidocaine is described in Examples 4-7.

### Utility

The method for treatment of asthma and the inhalable lidocaine compositions disclosed herein are suitable for treatment of both seasonal and severe and persistent asthma as well as for reducing or eliminating cortisteroid treatment simultaneously provided to asthmatic patients.

### EXAMPLE 1

### Lidocaine Solution for Inhalation

This example describes lidocaine solution for inhalation used for in vivo studies.

LSI is provided as a 1.0 mL sterile, preservative free, nonpyrogenic single dose ampule. The ampules contain 10 or 40 mg of lidocaine hydrochloride, USP (1 mL 1% or 4% of lidocaine hydrochloride solution), in a pH range of 5.0 to 7.5. The added sodium chloride content is 6.844 g/L of sodium chloride, USP for 1% lidocaine and 0.351 g/L of sodium chloride, USP for 4% lidocaine. The osmolality for both solutions is approximately 275-300 mOsm/kg.

LSI is intended for use in combination with the PARI eFlow nebulizer. It is not to be used for topical, percutaneous injection, spinal or regional nerve infiltration, or intravenous administration. For dosing instructions, see the study specific protocol.

### EXAMPLE 2

### Preparation of Lidocaine Dry Powder

This example provides methods and procedures used for preparation of lidocaine containing inhalable dry powder.

For dry powder formulation of the invention, a purified lidocaine is processed to a powder having mass median average diameters ranging from 3 ìm to 10 μm by media milling, jet milling, spray drying, or particle precipitation techniques.

Media milling may be accomplished by placing lidocaine substance into a mill containing, for example, stainless steel or ceramic balls and rotating or tumbling the material until the desired drug particle size ranges are achieved.

Jet milling uses very high pressure air streams to collide particles with one another, with fine particles of the desired size being recovered from the mill.

Spray drying is achieved by spraying a fine mist of lidocaine solution onto a support and drying the particles. The particles are then collected.

Particle precipitation is achieved by adding a co-solvent to spray dried particles. The solubility of the drug falls to the point where solid drug particles are formed. The particles are collected by filtration through 3 ìm filter or centrifugation. Precipitation has the advantage of being highly reproducible and can be performed under low temperature conditions, which reduce degradation.

### EXAMPLE 3

### Dry Powder Inhalators for Lidocaine Dry Powder

The lidocaine dry powder formulations of the invention may be used directly in metered dose or dry powder inhalers.

A metered dose inhaler consists of three components: a canister containing the propellant lidocaine suspension, a metering valve designed to deliver accurately metered volumes of the propellant suspension, and an oral adapter which contains a spray orifice from which the metered dose is delivered. In the rest position, the metering chamber of the valve is connected to the drug suspension reservoir via a filling groove or orifice. On depression of the valve this filling groove is sealed and the metering chamber is exposed to atmospheric pressure via the spray orifice in the oral adapter and the valve stem orifice. This rapid pressure reduction leads to flash boiling of the propellant and expulsion of the rapidly expanding mixture from the metering chamber. The liquid/vapor mixture then enters the expansion chamber which is constituted by the internal volume of the valve stem and the oral adapter. The mixture undergoes further expansion before being expelled, under its own pressure, from the spray nozzle. On exit from the spray orifice, the liquid ligaments which are embedded in propellant vapor are torn apart by aerodynamic forces. Typically, at this stage, the droplets are 20 to 30μ in diameter and are moving at the velocity of sound of the two-phase vapor liquid mixture (approximately 30 meters per second). As the cloud of droplets moves away from the spray nozzle, it entrains air from the surroundings and decelerates, while the propellant evaporates through evaporation, the entrained droplets eventually reach their residual diameter.

At this point, the particles/droplets consist of a powdered lidocaine core coated with surfactant. Depending on the concentration and the size of the suspended material the powdered drug core consists of either individual drug particles or aggregates. Currently, meter dose inhaler technology is optimized to deliver masses of 80 to 100 micrograms of drug, with an upper limitation of 1 mg of drug deliverable.

An alternated route of lidocaine dry powder delivery is by dry powder inhalers. Excipients commonly used are lactose, however in the case of lidocaine free base the addition of the amino acids lysine or leucine will lead to better powder formation.

Effective dosage levels of lidocaine for dry powder inhalation and metered dose inhalation result in the application of a nominal dose of at least about 10 mg, and more preferable about 40 mg of lidocaine to the conducting and central airways of the patient receiving treatment. Deposited dose are 2 and 20 mg in the conducting and central airways for 10 mg and 40 mg nominal dose, respectively. Depending on the efficiency of the dry powder delivery device, dry powder formulations suitable for use in the invention comprise from about 1.0 to about 50 mg, preferably from about 10 to about 40 mg of powder in an amorphous or crystalline lidocaine in particle sizes between 3 ìm and 10 microns in mass median average diameter necessary for efficacious delivery of lidocaine into the central airways. The dry powder formulation may be delivered from 1 to 4 times daily, preferably twice daily, for a period of at least one day, more preferably at least 5 days and most preferably at least fourteen days or longer, typically for as long as asthma persists. The dry powder formulations are temperature stable and have a physiologically acceptable pH of 5.0 to 7.5, preferably 5.5 to 7.0, and long shelf lives.

### EXAMPLE 4

### Clinical Trial for Treatment of Asthma

This example illustrates a clinical trial with inhalable lidocaine solution (10 and 40 mg) for treatment of asthma.

The clinical trial was performed in a double blinded, placebo controlled study in mild to moderate asthma patients. For the study, 10 mg (1 ml of 1% lidocaine/saline), 40 mg (1 ml of 4% lidocaine/saline) of lidocaine solution for inhalation or placebo (1 ml of saline) was administered by the electronic nebulizer PARI eFlow, modified, twice daily.

Asthma patients (100 females and 54 males, 31.3±1.8 years of age, FEV₁ 78.4±1.8% predicted) were enrolled, randomized to three groups, and treated for 12 weeks. The full individual doses of 1 ml were administered in 2-3 minutes treatment time.

Both doses of lidocaine were found to be well tolerated and safe, and no difference was found in the number of patients with at least one adverse events (AEs) were found between placebo (30/48, 63%), 10 mg lidocaine (39/55, 71%), and 40 mg lidocaine (33/51, 65%). Particularly, there was no difference in the number of patients with one or more respiratory AEs between placebo (14/48, 29%), 10 mg lidocaine (16/55, 29%), and 40 mg lidocaine (18/51, 35%).

Airway irritation and acute bronchospasm were assessed by measuring spirometry immediately prior to and 30 min post-completion of aerosol administration. A decrease in forced expired volume in one second (FEV1) >20% in the 30 minutes spirometry test was considered evidence of bronchospasm. All patients were tested for bronchospasm upon aerosolization of all three doses mentioned above (LSI 1%, 4%, and placebo) , and FEV1 was compared before and after drug application of drug. None of the 154 treated had occurrence of bronchospasm.

The amount of complaints about oropharyngeal numbing was greatly reduced when compared to previous studies with inhaled lidocaine given by other nebulizers. Numbing in all previous studies had been noted in virtually all subjects, whereas the incidence of numbing in this clinical trial was 40% (22/55) and 31.45% (16/51) of treated patients, noted at least once during the course of the 12 week study and 60% in 1% group and 68.6% in 4% group of lidocaine treated patients did not have any complaints of numbing at all.

No loss of gag reflex was reported. No case of tracheal aspiration of food or liquid was reported. Most importantly, there were no reports of bronchospasm upon lidocaine delivery, nor was there any report of a clinically significant FEV₁drop upon lidocaine inhalation (the measure for bronchospasm).

Within the Asthma Quality of Life Questionnaires (AQLQ) (environmental domain), all patients were asked about how they tolerated impact of dust, smoke, and air pollution. When AQLQs were analyzed, a significant improvement was observed at week 12 from the baseline between treatment groups and placebo in the environmental domain of the questions (mean changes were placebo=0.35; 10 mg lidocaine=2.09; and 40 mg lidocaine=2.45; p=0.012 for 10 mg lidocaine v. placebo, p=0.01 for 40 mg v. placebo).

These results indicate a post-treatment an improvement of airway susceptibility and asthma in tolerating environmental airway challenges like smoke, dust, pollution, allergens, etc.

The lung deposition of lidocaine is increased two- to three-fold. The same efficacious amount can be delivered in ⅓ of the time used previously. The safety profile is greatly improved, with abolished bronchospasm, greatly reduced incidence of oropharyngeal numbing, loss of gag reflex with lower lung and systemic deposition of lidocaine.

These results confirm that the inhlable lidocaine administered according to the invention is safe and tolerable upon its delivery to the conducting airways. None of the descriptions of prior art provided a convenient, tolerable and safe administration of lidocaine to the upper and central airways.

### EXAMPLE 5

### Treatment of Asthma Clinical Trial II—Case Report

This example describes a case report of treatment for asthma in a male patient using 1% of lidocaine.

A 38 year old, otherwise healthy male patient had experienced postnasal drip and persistent asthma for two weeks, after having had an upper respiratory infection. Asthma episodes were occurring hourly during the day, and were influencing the patients' sleep at night.

Lidocaine 1% (25 mg/l ml saline) was administered as a single dose via electronic PARI eFlow nebulizer. During the treatment (approx. 2-3 minutes) and after the treatment no significant numbing of the oropharynx nor loss of gag reflex was reported, and only a transient numbing of the tongue was noted. The patient reported good tolerability, and no otherwise untoward effects. After this single treatment, the patients' asthma was greatly diminished over the course of the following day, and the patient remained essentially free of asthma for the following two weeks.

### EXAMPLE 6

### Treatment of Moderate Severity Asthma with Lidocaine Solution for Inhalation

This example describes efficacy of the 1% (10 mg/ml) aerosolized lidocaine solution for treatment of moderate asthma in asthmatic patients.

The effect of lidocaine solution for inhalation (LSI) was explored in a clinical study involving mild asthmatics. The clinical goal of treatment with LSI in the targeted population was a reduction of the inflammatory process and improvement in pulmonary function and other clinical parameters that reflect the burden of asthma. This study was designed to investigate the therapeutic potential of 1% or 4% aerosolized lidocaine solution in subjects with moderate asthma who were steroid-naive but required β-agonist rescue treatment.

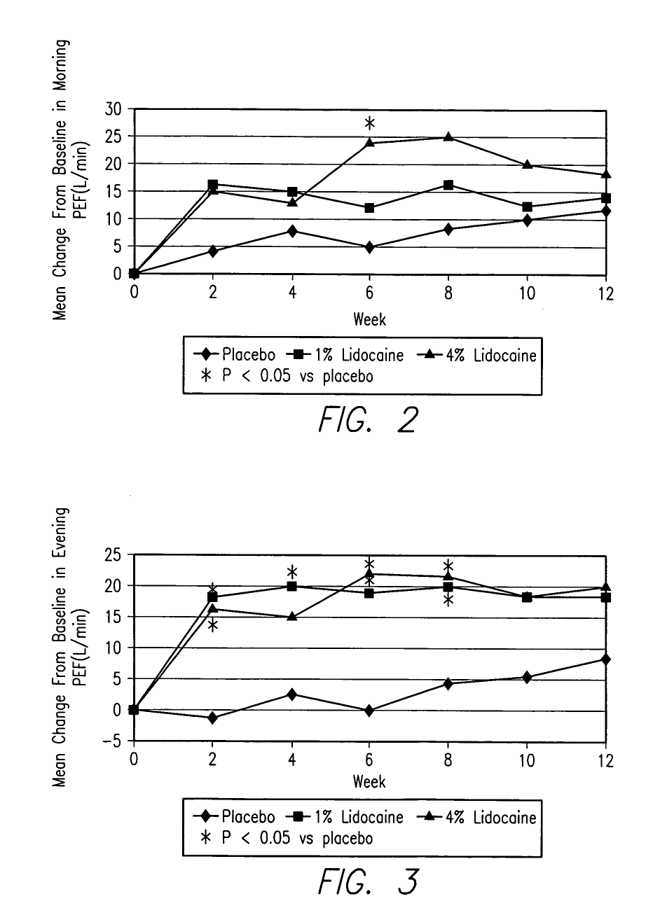

Asthma subjects were 12 years or older with FEV₁ ≧60% of predicted and 12% FEV₁ reversibility. Subject were treated either with placebo (1 ml of saline), 1% (10 mg/ml) or 4% (40 mg/ml) lidocaine solution for inhalation having pH between 5.5 and 7, for twelve weeks. Mean change from baseline morning and evening peak expiratory flow was determined and is shown in FIGS. 2 and 3.

Exploratory analysis on percent change in FEV₁ in subjects with baseline FEV1 <70% predicted showed mean change of 6.1% improvement at week 12 in the 1% LSI group. However, the same pattern did not hold for the 4% LSI group where these changes were lower. Area under the curve (AUC) analysis of morning (AM) peak expiratory flow, seen in FIG. 2 and evening (PM) seen in FIG. 3, showed a clinically meaningful improvement in both lidocaine groups of approximately 20 L/min. The observed improvement in peak flow is comparable to improvement in peak expiratory flow seen with leukotriene-inhibitors. The benefit is larger in the moderate asthma subjects indicating that 1% LSI is an efficacious treatment of subjects whose asthma is of moderate severity.

Similarly, AQLQ showed a clinically meaningful trend for improvement, which approached statistical significance. Mean baseline total MiniAQLQ scores ranged from 66 to 68 for the three treatment groups. The week 12 mean change from Baseline was 4.2 for placebo, 10.2 for 1% lidocaine (p=0.0591 versus placebo), and 9.3 for 4% lidocaine (p=0.1148 versus placebo). The trend for AQLQ improvement was present in both lidocaine groups (1 and 4%) regardless of baseline disease severity. The placebo-adjusted improvement was larger in the moderate asthma severity subjects. The improvement seen was most pronounced in the environmental domain (tolerance to dust, allergens or air pollution and cigarette smoke) which showed significant improvement in both lidocaine groups.

### EXAMPLE 7

### Treatment of Asthma with Reducing the Need for a Concurrent Treatment with Corticosteroids

This example describes a study performed for determination of efficacy of lidocaine for inhalation solution in reducing or eliminating a need for concurrently administered corticosteroids to patients with steroid dependent severe asthma.

The safety, efficacy and tolerability of lidocaine solution for inhalation are currently studied in subjects with severe asthma who are being treated with oral corticosteroids. The main goal of the study is to wean patients from oral corticosteroids completely or reduce the oral corticosteroid dose by at least 50% and to improve clinical parameters related to asthma.

The primary efficacy endpoint is to monitor the efficacy of nebulized lidocaine in subjects by determining the total number of subjects able to reduce their oral corticosteroid dose by ≧50% and remain clinically stable for more than 4 weeks after the end of the treatment (week 20).

The therapeutic potential of 4% LSI for reduction or elimination of need for the concurrent treatment with corticosteroids is studied in patients diagnosed with asthma for ≧6 months, who have been and are treated with oral corticosteroids for at least six months and currently use, at a minimum, 5 mg or more of prednisone per day and used this dose during the previous 30 days.

The duration of treatment is 24 weeks, with a 2-week screening period, a 2-week treatment and tolerability period and an 18-week treatment period which includes a 14-week oral corticosteroids (OCS) tapering period.

At this time, 79 patients are in the tapering phase of the study, and 18 patients have completed the study. All patients have completed at least the halfway point of the study (visit 7) with 5 adverse effects reported. None of the reported adverse events were thought to be related to the lidocaine treatment. Reported adverse effects were migraine, eye occlusion (cataracts) and exacerbation of asthma.

Up-to-date, obtained results of studied 1% or 4% lidocaine for inhalation show that, using the method of the invention comprising an appropriate lidocaine formulation, nominal amount of lidocaine administered twice daily in a minimal amount of saline combined with a specifically modified electronic nebulizer, lidocaine can be safely and efficiently delivered to human lungs for treatment of asthma.

What is claimed is:

1. A method for treatment of asthma, said method comprising steps:
a) preparing an inhalable composition comprising either about 10 mg or about 40 mg of lidocaine;
b) selecting an electronic nebulizer, dry powder inhaler or metered dose inhaler able to generate aerosol of particle sizes substantially between 3.0 and 10 μm;
c) nebulizing said lidocaine formulation into an aerosol having a mass median aerodynamic diameter of particles substantially between about 3 μm and about 10 μm having a geometric standard deviation lower than 1.7;
d) administering said aerosolized formulation to a patient suffering from asthma.

2. The method of claim 1 wherein lidocaine is formulated as a solution for inhalation and is dissolved in about 1-3 ml of normal or diluted saline.

3. The method of claim 2 wherein the saline is of a normal strength.

4. The method of claim 2 wherein the saline is diluted to from 1/10 to 9/10 normal strength.

5. The method of claim 2 wherein lidocaine is administered one to four times a day.

6. The method of claim 5 wherein lidocaine is formulated as a solution for inhalation comprising 10 mg or 40 mg of lidocaine per one ml of saline having pH adjusted to between 5.5 and 7.0, osmolality to between about 275 and 300 mOsm/kg, viscosity to about 1.5 centipoise and a permeant anion concentration between 31 and 30 mM, wherein said solution is free of preservatives.

7. The method of claim 6 wherein lidocaine for inhalation administered by nebulization using an electronic nebulizer.

8. The method of claim 7 wherein the nebulizer is a PARI eFlow” electronic nebulizer.

9. The method of claim 8 wherein the PARI eFlow™ electronic nebulizer is modified to comprise a vibrating perforate membrane.

10. The method of claim 1 where the lidocaine formulation is sterilized and packaged in a low density polyethylene sealed vial under sterile conditions for storage.

11. The method of claim 1 wherein lidocaine is formulated as a dry powder.

12. The method of claim 11 wherein the lidocaine dry powder is delivered by a dry powder inhaler or by a metered dose inhaler.

13. The method of claim 7 wherein lidocaine dry powder is administered one to four times a day by nebulization in an aerosol having MMAD between 3.5 and 10 μm.

14. A method of treatment of severe asthma and for decreasing a need for corticosteroids treatment in asthmatic patients being concurrently treated with corticosteroids, said method comprising steps:
a) preparing an inhalable formulation comprising about 40 mg of lidocaine;
b) selecting an electronic nebulizer, dry powder or meter dose inhaler able to generate aerosol of particle sizes substantially between 3.0 and 10 μm;
c) nebulizing said lidocaine formulation into an aerosol having a mass median aerodynamic diameter of particles substantially between about 3 μm and about 10 μm having a geometric standard deviation lower than 1.7;
d) determining a pretreatment value for a forced expiratory volume per one second, airway irritation, acute bronchospasm and clinical stability of the patient suffering from asthma;
e) withdrawing or decreasing a dose of administered oral or inhaled corticosteroids in the treated patient;
f) administering said aerosolized formulation to the patient; and
g) determining a post-treatment value or decrease in the value for a forced expiratory volume per one second after the treatment and in set intervals during and following the treatment.

15. The method of claim 14 wherein lidocaine is formulated as a solution for inhalation and dissolved in about 1-3 ml of normal or diluted saline.

16. The method of claim 15 wherein the saline is of a normal strength.

17. The method of claim 15 wherein the saline is diluted to from 1/10 to 9/10 normal strength.

18. The method of claim 15 wherein lidocaine is administered one to four times a day.

19. The method of claim 18 wherein lidocaine is formulated as a solution for inhalation comprising 10 mg or 40 mg of lidocaine per one ml of saline having pH adjusted to between 5.5 and 7.0, osmolality to between about 275 and 300 mOsm/kg, viscosity to about 1.5 centipoise and a permeant anion concentration between 31 and 300 mM, wherein said solution is free of preservatives.

20. The method of claim 19 wherein lidocaine for inhalation is administered by nebulization using an electronic nebulizer.

21. The method of claim 20 wherein the nebulizer is a PARI eFlow™ electronic nebulizer.

22. The method of claim 21 wherein the PARI eFlow™ electronic nebulizer is modified to comprise a vibrating perforate membrane.

23. The method of claim 14 wherein lidocaine is formulated as a dry powder.

24. The method of claim 23 wherein the lidocaine dry powder is delivered by a dry powder inhaler or by a metered dose inhaler.

25. The method of claim 24 wherein lidocaine dry powder is administered one to four times a day as an aerosol having a MMAD between 3 and 10 μm.

26. An inhalable lidocaine composition comprising about 10 or about 40 mg of a lidocaine suitable for treatment of asthma prepared as an inhalable dry powder or as an aerosolable solution.

27. The composition of claim 26 wherein the lidocaine is formulated as an inhalable solution comprising from about 10 to about 40 mg or lidocaine dissolved in about 1 ml of saline and nebulized into an aerosol having a mass median diameter between about 4 μm and about 5 μm administered to an asthmatic patient one to four times a day.

28. The composition of claim 26 wherein the lidocaine is formulated as a dry powder.

29. The composition of claim 28 wherein the dry powder is prepared by milling, spray drying or particle precipitation to the powder having a particle size with a mass median aerodynamic diameter from about 1 μm to about 5 μm.

30. The composition of claim 29 wherein the powder additionally comprises an excipient particle.

31. The composition of claim 30 wherein the excipient particle is lactose, lysine or leucine.
